(19) United States
(12) Patent Application Publication
(10) Pub. No.: US 2023/0149466 A1
(43) Pub. Date: May 18, 2023

(54) IMMUNOTHERAPEUTIC METHODS AND COMPOSITIONS FOR TARGETING CANCER FIBROBLASTS

(71) Applicant: FIGENE, LLC, Houston, TX (US)

(72) Inventor: Thomas ICHIM, San Diego, CA (US)

(21) Appl. No.: 17/995,609

(22) PCT Filed: Apr. 30, 2021
(86) PCT No.: PCT/US2021/070496

§ 371 Date: Oct. 6, 2022

Related U.S. Application Data
(60) Provisional application No. 63/017,918, filed on Apr. 30, 2020.

(51) Int. Cl. A61K 35/17; A61K 35/51; A61K 45/06; C12N 5/0783

(52) U.S. Cl. CPC A61K 35/17; A61K 35/51; A61K 45/06; C12N 5/0646; C12N 2501/51; C12N 2501/065; C12N 2501/515; C12N 2501/2302

(57) ABSTRACT

Aspects of the present disclosure are directed to methods and compositions for inducing the generation of cells capable of killing cancer associated fibroblasts at least in part through the generation of ex vivo expanded cord blood cells which are programmed to kill cancer associated fibroblasts. Certain aspects relate to generation of immune cells, including T cell and Natural Killer (NK) cells, for use in targeting cancer cells and cancer associated fibroblasts. Further aspects are directed to gene editing and/or gene silencing of immune checkpoint proteins in therapeutic immune cells.

This application claims priority to U.S. Provisional Pat. Application Serial No. 63/017,918, filed Apr. 30, 2020, which is incorporated by reference herein in its entirety.

### TECHNICAL FIELD

Embodiments of the disclosure encompass at least the fields of immunology, cancer biology, and medicine.

### BACKGROUND

The immune system killing cancer was originally a controversial subject, however, recent studies support this notion. For example, the incidence of spontaneous cancer is substantially higher in mice lacking NK cell activity [1-4]. In human studies, it has been shown that patients with higher tumor infiltrating lymphocytes possess longer survival [5-7].

Cancer suppresses the immune system in part through inducing expansion of activity and number of T regulatory cells, whose normal function in the body is controlling the pathological immunity such as in autoimmune situations [8-10][11]. Studies have shown that T regulatory cells suppress activity of various cancer therapeutics [12][13][14]. One method of overcoming cancer immune suppression is to destroy the ability of the cancer to suppress the immune system.

Although progress has been made in extending patient’s lives, significant hurdles exist in terms of the patients that do not respond to therapy, or where responses are short lived. There is a need for improved methods for treating cancer, including by preventing cancer from suppressing the immune system.

### BRIEF SUMMARY

Aspects of the present disclosure are directed to methods for inducing an immune response to cancer-associated fibroblasts. Further aspects are directed to engineered cord blood-derived cells, including natural killer cells, and their use in killing or eliminating cancer-associated fibroblasts.

Disclosed herein, in some embodiments, is a method of inducing an immune response to cancer-associated fibroblasts in a subject, the method comprising: (a) isolating mononuclear cells from a cord blood sample; (b) culturing the mononuclear cells with cancer-associated fibroblasts and one or more factors capable of enhancing cytotoxic activity of the mononuclear cells; (c) inhibiting expression of an immune checkpoint protein in the mononuclear cells; and (d) administering the mononuclear cells to the subject. In some embodiments, the cancer-associated fibroblasts have been mitotically inactivated. In some embodiments, (c) comprises RNA interference. In some embodiments, (c) comprises providing to the mononuclear cells an antisense oligonucleotide capable of inhibiting expression of the immune checkpoint protein. In some embodiments, (c) comprises providing to the mononuclear cells a short interfering RNA (siRNA) or short hairpin RNA (shRNA) capable of inhibiting expression of the immune checkpoint protein. In some embodiments, the immune checkpoint protein is NR2F6, PD-1, PD-1L, TIM-3, CTLA-4, CD200, STAT6, or indolamine 2,3, deoxygenase (IDO). In some embodiments, the immune checkpoint protein is PD-1, CTLA-4, or TIM-3. In some embodiments, the one or more factors comprise one or more of IL-2, anti-CD3 antibodies, anti-CD28 antibodies, IL-7, IL-12, IL-17, IL-15, IL-18, and IL-33. In some embodiments, the one or more factors comprise IL-2. In some embodiments, the one or more factors comprise anti-CD3 antibodies and anti-CD28 antibodies.

In some embodiments, the one or more factors comprise a toll-like receptor (TLR) agonist. In some embodiments, the TLR agonist is a TLR-1 agonist. In some embodiments, the TLR-1 agonist is Pam3CSK4. In some embodiments, the TLR agonist is a TLR-2 agonist. In some embodiments, the TLR-2 agonist is heat-killed Listeria monocytogenes (HKLM). In some embodiments, the TLR agonist is a TLR-3 agonist. In some embodiments, the TLR-3 agonist is Poly I:C. In some embodiments, the TLR agonist is a TLR-4 agonist. In some embodiments, the TLR-4 agonist is Buprenorphine. In some embodiments, the TLR-4 agonist is Carbamazepine. In some embodiments, the TLR-4 agonist is Fentanyl. In some embodiments, the TLR-4 agonist is Levorphanol. In some embodiments, the TLR-4 agonist is Methadone. In some embodiments, the TLR-4 agonist is Cocaine. In some embodiments, the TLR-4 agonist is Morphine. In some embodiments, the TLR-4 agonist is Oxcarbazepine. In some embodiments, the TLR-4 agonist is Oxycodone. In some embodiments, the TLR-4 agonist is Pethidine. In some embodiments, the TLR-4 agonist is Glucuronoxylomannan from Cryptococcus. In some embodiments, the TLR-4 agonist is Morphine-3-glucuronide. In some embodiments, the TLR-4 agonist is lipoteichoic acid. In some embodiments, the TLR-4 agonist is β-defensin 2. In some embodiments, the TLR-4 agonist is small molecular weight hyaluronic acid. In some embodiments, the TLR-4 agonist is fibronectin EDA. In some embodiments, the TLR-4 agonist is snapin. In some embodiments, the TLR-4 agonist is tenascin C. In some embodiments, the TLR agonist is a TLR-5 agonist. In some embodiments, the TLR-5 agonist is flagellin. In some embodiments, the TLR agonist is a TLR-6 agonist. In some embodiments, the TLR-6 agonist is FSL-1. In some embodiments, the TLR agonist is a TLR-7 agonist. In some embodiments, the TLR-7 agonist is imiquimod. In some embodiments, the TLR agonist is a TLR-8 agonist. In some embodiments, the TLR-8 agonist is ssRNA40/LyoVec. In some embodiments, the TLR agonist is a TLR-9 agonist. In some embodiments, the TLR-9 agonist is a CpG oligonucleotide. In some embodiments, the TLR-9 agonist is ODN2006. In some embodiments, the TLR-9 agonist is Agatolimod.

In some embodiments, (b) comprises culturing the mononuclear cells and the cancer-associated fibroblasts with an antigen presenting cell. In some embodiments, the antigen presenting cell is a dendritic cell. In some embodiments, the dendritic cell is a myeloid dendritic cell. In some embodiments, the dendritic cell is a lymphoid dendritic cell. In some embodiments, the antigen presenting cell is a B cell. In some embodiments, the antigen presenting cell is a neutrophil. In some embodiments, the antigen presenting cell is an endothelial cell. In some embodiments, the antigen presenting cell is an artificial antigen presenting cell. In some embodiments, the antigen presenting cell enhances immunogenicity of the mononuclear cells. In some embodiments, the antigen presenting cell enhances immunogenicity of the mononuclear cells by augmenting HLA expression. In some embodiments, the antigen presenting cell enhances immunogenicity of the mononuclear cells by augmenting TAP expression. In some embodiments, the antigen presenting cell enhances immunogenicity of the mononuclear cells by augmenting CD80 expression. In some embodiments, the antigen presenting cell enhances immunogenicity of the mononuclear cells by augmenting CD86 expression. In some embodiments, the antigen presenting cell enhances immunogenicity of the mononuclear cells by augmenting IL-12 expression. In some embodiments, (b) comprises culturing the mononuclear cells and the cancer-associated fibroblasts with a soluble inhibitor of one or more immunosuppressive factors. In some embodiments, the soluble inhibitor is a small molecule. In some embodiments, the soluble inhibitor is an antibody. In some embodiments, the one or more immunosuppressive factors comprise one or more of HLA-G, IL-10, IDO, cyclo-oxygenases, and IL-20. In some embodiments, prior to (d), a chemotherapeutic agent is administered to the subject. In some embodiments, the chemotherapeutic agent is a small molecule, a nucleic acid, an antibody, or an antibody-like molecule. In some embodiments, the chemotherapeutic agent is ifosfamide, nimustine hydrochloride, cyclophosphamide, dacarbazine, melphalan, and ranimustine, gemcitabine hydrochloride, enocitabine, cytarabine ocfosfate, a cytarabine formulation, tegafur/uracil, a tegafur/gimeracil/oteracil potassium mixture, doxifluridine, hydroxycarbamide, fluorouracil, methotrexate, mercaptopurine, idarubicin hydrochloride, epirubicin hydrochloride, daunorubicin hydrochloride, daunorubicin citrate, doxorubicin hydrochloride, pirarubicin hydrochloride, bleomycin hydrochloride, peplomycin sulfate, mitoxantrone hydrochloride, mitomycin C, etoposide, irinotecan hydrochloride, vinorelbine tartrate, docetaxel hydrate, paclitaxel, vincristine sulfate, vindesine sulfate, vinblastine sulfate, anastrozole, tamoxifen citrate, toremifene citrate, bicalutamide, flutamide, estramustine phosphate, carboplatin, cisplatin, nedaplatin, thalidomide, neovastat, and bevacizumab, or L-asparaginase.

In some embodiments, the mononuclear cells are capable of inducing an immunological response in the subject toward the cancer-associated fibroblasts. In some embodiments, the immunological response is cell proliferation. In some embodiments, the immunological response is expression of CD69. In some embodiments, the immunological response is expression of CD25. In some embodiments, the immunological response is expression of perforin. In some embodiments, the immunological response is expression of granzyme. In some embodiments, the immunological response is expression of Fas ligand. In some embodiments, the immunological response is expression of a cytokine that suppresses proliferation of tumor associated fibroblasts. In some embodiments, the cytokine induces cell cycle arrest of tumor associated fibroblasts cells. In some embodiments, the cytokine induces apoptosis of tumor associated fibroblast cells. In some embodiments, the cytokine induces autophagy of tumor associated fibroblast cells. In some embodiments, the cytokine induces necrosis of tumor associated fibroblast cells. In some embodiments, the cytokine is interferon gamma. In some embodiments, the cytokine is TNF-alpha, interferon alpha, interferon beta, interferon gamma, interferon omega, interferon tau, TRAIL, IL-2, IL-7, IL-12, IL-17, IL-18, IL-21, IL-22, IL-23, IL-27, IL-33, or HMGB1.

In some embodiments, the mononuclear cells are immune cells. In some embodiments, the immune cells are natural killer (NK) cells. In some embodiments, the immune cells are natural killer T (NKT) cells. In some embodiments, the immune cells are T cells. In some embodiments, the method further comprises providing to the T cells a cancer-associated fibroblast antigen. In some embodiments, the cancer-associated fibroblast antigen is fibroblast activated protein 1, FGF-9, TEM, VEGFR2, NA17, PDGFR-β, PAP, MAD-CT-2, Tie-2, PSA, protamine 2, legumain, endosialin, prostate stem cell antigen, carbonic anhydrase IX, STn, proteinase 3, GM3 ganglioside, or EpCAM. In some embodiments, the T cells possess a Th1 phenotype. In some embodiments, the T cells express one or more of CD4, CD94, CD119 (IFN_{γ} R1), CD183 (CXCR3), CD186 (CXCR6), CD191 (CCR1), CD195 (CCR5), CD212 (IL-12Rβ1&2), CD254 (RANKL), CD278 (ICOS), IL-18R, MRP1, NOTCH3, and TIM3.

Disclosed herein, in some embodiments, is method of killing cancer-associated fibroblasts comprising culturing the cancer-associated fibroblasts with activated natural killer (NK) cells. In some embodiments, the activated NK cells were obtained from cord blood. In some embodiments, the activated NK cells comprise a genetic modification, wherein the genetic modification reduces the expression of an immune checkpoint protein. In some embodiments, the immune checkpoint protein is NR2F6, PD-1, PD-1L, TIM-3, CTLA-4, CD200, STAT6, or indolamine 2,3, deoxygenase (IDO). In some embodiments, the NK cells were activated using culture with IL-2.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

FIG. 1 presents results from the studies described in Example 1, showing a graph of percent cytotoxicity as assessed by chromium release at the indicated ratios of CD56+ NK cells to fibroblasts (1:1, 5:1, or 10:1). Shown from left to right are results from control fibroblasts only, cancer-associated fibroblasts only, NK cells cultured with control fibroblasts, and NK cells cultured with cancer-associated fibroblasts.

### DETAILED DESCRIPTION

One method of overcoming cancer immune suppression is to destroy the ability of the cancer to suppress the immune system. One possible means of doing this is by attacking cells that protect the cancer, in particularly, cancer associated fibroblasts present a potentially valuable target for immunotherapy. Since the cancer associated fibroblasts act like a “shield” for the tumor, using the immune system to attack these cells presents a new option.

The first widespread utilization of cord blood as a stem cell source was in the treatment of pediatric hematological malignancies after myeloablative conditioning. Since matching requirements for this type of transplant are not as strict as for hematopoietic stem cell sources, cord blood began gaining acceptance in adult patients lacking bone marrow donors [16-21]. Outside the area of oncology, the clinical use of cord blood has expanded into various areas that range from reconstituting a defective immune system [22], to correcting congenital hematological abnormalities [23], to inducing angiogenesis [24]. To our knowledge cord blood has not been used in an allogeneic setting following gene silencing/gene editing of immunological checkpoints.

The present disclosure includes methods comprising administering allogeneic cord blood-derived cells have been gene silenced or permanently gene edited so as to not succumb to tumor inhibition. For example, in some embodiments, disclosed are methods comprising silencing or deleting immune checkpoint proteins (e.g., PD1, CTLA4, etc.) from cord blood-derived cells and use of such cells for targeting cancer-associated fibroblasts. Furthermore, in some embodiments, gene edited cord-blood derived lymphocytes possess a suicide gene, which allows for destruction of the modified lymphocytes should autoimmunity or pathological consequences arise.

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

As used herein, “allogeneic” refers to tissues or cells or other material from another body that in a natural setting are immunologically incompatible or capable of being immunologically incompatible, although from one or more individuals of the same species.

As used herein, “cell line” refers to a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to seeding density, substrate, medium, growth conditions, and time between passaging.

As used herein, “conditioned medium” describes medium in which a specific cell or population of cells has been cultured for a period of time, and then removed, thus separating the medium from the cell or cells. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. In this example, the medium containing the cellular factors is conditioned medium.

As used herein, a “trophic factor” describes a substance that promotes and/or supports survival, growth, proliferation and/or maturation of a cell. Alternatively or in addition, a trophic factor stimulates increased activity of a cell.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “reduced” or “reduce” as used herein generally means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease, or any integer decrease between 10-100% as compared to a reference level.

The term “increased” or “increase” as used herein generally means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any integer increase between 10-100% as compared to a reference level, or about a 2-fold, or about a 3-fold, or about a 4-fold, or about a 5-fold or about a 10-fold increase, or any increase between 2-fold and 10-fold or greater as compared to a reference level.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As one example, an effective amount is the amount sufficient to reduce immunogenicity of a group of cells. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

As used herein, the terms “treatment,” “treat,” or “treating” refers to intervention in an attempt to alter the natural course of the individual or cell being treated, and may be performed either for prophylaxis or during the course of pathology of a disease or condition. Treatment may serve to accomplish one or more of various desired outcomes, including, for example, preventing occurrence or recurrence of disease, alleviation of symptoms, and diminishment of any direct or indirect pathological consequences of the disease, preventing metastasis, lowering the rate of disease progression, amelioration or palliation of the disease state, and remission or improved prognosis.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

A variety of aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

The term “subject,” as used herein, may be used interchangeably with the term “individual” and generally refers to an individual in need of a therapy. The subject can be a mammal, such as a human, dog, cat, horse, pig or rodent. The subject can be a patient, e.g., have or be suspected of having or at risk for having a disease or medical condition related to bone. For subjects having or suspected of having a medical condition directly or indirectly associated with bone, the medical condition may be of one or more types. The subject may have a disease or be suspected of having the disease. The subject may be asymptomatic. The subject may be of any gender. The subject may be of a certain age, such as at least 30, 35, 40, 45, 50, 55, 60, 65, 70, 75, 80, 85, 90, 95, or 100 or more.

The term “fibroblast-derived product” (also “fibroblast-associated product”), as used herein, refers to a molecular or cellular agent derived or obtained from one or more fibroblasts. In some cases, a fibroblast-derived product is a molecular agent. Examples of molecular fibroblast-derived products include conditioned media from fibroblast culture, microvesicles obtained from fibroblasts, exosomes obtained from fibroblasts, apoptotic vesicles obtained from fibroblasts, nucleic acids (e.g., DNA, RNA, mRNA, miRNA, etc.) obtained from fibroblasts, proteins (e.g., growth factors, cytokines, etc.) obtained from fibroblasts, and lipids obtained from fibroblasts. In some cases, a fibroblast-derived product is a cellular agent. Examples of cellular fibroblast-derived products include cells (e.g., stem cells, hematopoietic cells, neural cells, etc.) produced by differentiation and/or de-differentiation of fibroblasts.

The term “NK cells,” as used herein, refers to CD3-negative/CD56-positive mononuclear cells. The disclosed NK cells have cytotoxic activity against cells in which expression of MHC class I molecules is reduced or the expression is lost. In some embodiments, disclosed is the utilization of expanded NK cells for killing cancer associated fibroblasts.

The term “umbilical cord blood” as used herein refers to both a fresh umbilical cord blood collected from an umbilical cord at the time of delivery and an umbilical cord blood in a frozen state available through an umbilical cord blood bank system in which an umbilical cord blood is cryopreserved after obtaining test data for histocompatibility.

“Binding” refers to a sequence-specific, non-covalent interaction between macromolecules. Not all components of a binding interaction need be sequence-specific (e.g., contacts with phosphate residues in a DNA backbone), as long as the interaction as a whole is sequence-specific.

The term “binding protein” is a protein that is able to bind to another molecule. A binding protein can bind to, for example, a DNA molecule (a DNA-binding protein), an RNA molecule (an RNA-binding protein) and/or a protein molecule (a protein-binding protein).

“CRISPR/Cas nuclease” or “CRISPR/Cas nuclease system” includes a non-coding RNA molecule (guide) RNA that binds to DNA and Cas proteins (Cas9) with nuclease functionality (e.g., two nuclease domains). Collectively, CRISPR system refers to transcripts and other elements involved in the expression of or directing the activity of CRISPR-associated (“Cas”) genes, including sequences encoding a Cas gene, a tracr (trans-activating CRISPR), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. A sequence or template that may be used for recombination into the targeted locus comprising the target sequences is referred to as an “editing template” or “editing polynucleotide” or “editing sequence”. In aspects of the invention, an exogenous template polynucleotide may be referred to as an editing template. In an aspect of the invention the recombination is homologous recombination.

“Cleavage” within the context of the current invention refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “guide sequence” is any polynucleotide sequence having sufficient complementarity with a target polynucleotide sequence to hybridize with the target sequence and direct sequence-specific binding of a CRISPR complex to the target sequence.

A “sequence” refers to a nucleotide sequence of any length, which can be DNA or RNA; can be linear, circular or branched and can be either single-stranded or double stranded. The term “donor sequence” refers to a nucleotide sequence that is inserted into a genome.

A “target site” or “target sequence” is a nucleic acid sequence that defines a portion of a nucleic acid to which a binding molecule will bind, provided sufficient conditions for binding exist. For example, the sequence 5′-GAATTC-3′ is a target site for the Eco RI restriction endonuclease.

“Checkpoint genes” (also “immune checkpoint genes”) are genes whose protein products are capable of inhibiting immune responses, for example immune response to cancer. Within the context of the disclosure, checkpoint genes include: a) the E3 ubiquitin ligase Cbl-b; b) CTLA-4; c) PD-1; d) TIM-3; e) killer inhibitory receptor (KIR); f) LAG-3; g) CD73; h) Fas; i) the aryl hydrocarbon receptor; j) Smad2; k) Smad4; 1) TGF-β receptor; and m) ILT-3.

“Nucleic acid,” “polynucleotide,” and “oligonucleotide” refers to a deoxyribonucleotide or ribonucleotide polymer, in linear or circular conformation, and in either single- or double-stranded form. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 116:281-297), comprises a dsRNA molecule.

An “expression vector” refers to a vector, plasmid or vehicle designed to enable the expression of an inserted nucleic acid sequence following transformation into the host. The cloned gene, i.e., the inserted nucleic acid sequence, is usually placed under the control of control elements such as a promoter, a minimal promoter, an enhancer, or the like. Initiation control regions or promoters, which are useful to drive expression of a nucleic acid in the desired host cell are numerous and familiar to those skilled in the art. Virtually any promoter capable of driving expression of these genes can be used in an expression vector, including but not limited to, viral promoters, bacterial promoters, animal promoters, mammalian promoters, synthetic promoters, constitutive promoters, tissue specific promoters, pathogenesis or disease related promoters, developmental specific promoters, inducible promoters, light regulated promoters; CYC1, HIS3, GAL1, GAL4, GAL10, ADH1, PGK, PHO5, GAPDH, ADC1, TRP1, URA3, LEU2, ENO, TPI, alkaline phosphatase promoters (useful for expression in Saccharomyces); AOX1 promoter (useful for expression in Pichia); β-lactamase, lac, ara, tet, trp, lP.sub.L, lP.sub.R, T7, tac, and trc promoters (useful for expression in Escherichia coli); light regulated-, seed specific-, pollen specific-, ovary specific-, cauliflower mosaic virus 35S, CMV 35S minimal, cassava vein mosaic virus (CsVMV), chlorophyll a/b binding protein, ribulose 1,5-bisphosphate carboxylase, shoot-specific, root specific, chitinase, stress inducible, rice tungro bacilliform virus, plant super-promoter, potato leucine aminopeptidase, nitrate reductase, mannopine synthase, nopaline synthase, ubiquitin, zein protein, and anthocyanin promoters (useful for expression in plant cells); animal and mammalian promoters known in the art including, but are not limited to, the SV40 early (SV40e) promoter region, the promoter contained in the 3′ long terminal repeat (LTR) of Rous sarcoma virus (RSV), the promoters of the E1A or major late promoter (MLP) genes of adenoviruses (Ad), the cytomegalovirus (CMV) early promoter, the herpes simplex virus (HSV) thymidine kinase (TK) promoter, a baculovirus 1E1 promoter, an elongation factor 1 alpha (EF1) promoter, a phosphoglycerate kinase (PGK) promoter, a ubiquitin (Ubc) promoter, an albumin promoter, the regulatory sequences of the mouse metallothionein-L promoter and transcriptional control regions, the ubiquitous promoters (HPRT, vimentin, -α-actin, tubulin and the like), the promoters of the intermediate filaments (desmin, neurofilaments, keratin, GFAP, and the like), the promoters of therapeutic genes (of the MDR, CFTR or factor VIII type, and the like), pathogenesis or disease related-promoters, and promoters that exhibit tissue specificity and have been utilized in transgenic animals, such as the elastase I gene control region which is active in pancreatic acinar cells; insulin gene control region active in pancreatic beta cells, immunoglobulin gene control region active in lymphoid cells, mouse mammary tumor virus control region active in testicular, breast, lymphoid and mast cells; albumin gene, Apo AI and Apo AII control regions active in liver, alpha-fetoprotein gene control region active in liver, alpha 1-antitrypsin gene control region active in the liver, beta-globin gene control region active in myeloid cells, myelin basic protein gene control region active in oligodendrocyte cells in the brain, myosin light chain-2 gene control region active in skeletal muscle, and gonadotropic releasing hormone gene control region active in the hypothalamus, pyruvate kinase promoter, villin promoter, promoter of the fatty acid binding intestinal protein, promoter of the smooth muscle cell α-actin, and the like. In addition, these expression sequences may be modified by addition of enhancer or regulatory sequences and the like.

An “siRNA” (also “small interfering RNA”) as used herein describes a nucleic acid that forms a double stranded RNA, which double stranded RNA has the ability to reduce or inhibit expression of a gene or target gene when the siRNA is expressed in the same cell as the gene or target gene. “siRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

### II. Methods and Compositions for Therapeutic Targeting of Cancer-Associated Fibroblasts

Aspects of the present disclosure provide methods and compositions for inducing the generation of cells capable of killing cancer associated fibroblasts at least in part through the generation of ex vivo expanded cord blood cells which are programmed to kill cancer associated fibroblasts.

In some embodiments of the disclosure, lymphocytes derived from umbilical cord blood are gene silenced or gene edited to reduce or completely abolish expression of checkpoint inhibitor genes while being expanded in vitro in the presence of cancer associated fibroblasts, or antigens derived from cancer associated fibroblasts. Described herein are compositions and methods for gene editing of checkpoint genes. In some embodiments, methods of the disclosure comprise the application of gene editing technology as a method for generating lymphocytes resistant to inhibitory signals. In further aspects, disclosed is the use of suicide genes to allow for deletion of manipulated lymphocytes administered to the host. Various methods for gene deletion or suppression of gene expression are contemplated and known to one skilled in the art.

Barrangou et al. [25] first demonstrated that clustered regularly interspaced short palindromic repeats (CRISPR) are found in the genomes of most Bacteria and Archaea and after bacteriophage challenge, the bacteria integrated new spacers derived from phage genomic sequences. Removal or addition of particular spacers modified the phage-resistance phenotype of the cell. They concluded that CRISPR, together with associated cas genes, provided resistance against phages, and resistance specificity is determined by spacer-phage sequence similarity. These techniques provided a clue that editing or deleting DNA segments may be possible. In 2013, Mali et al took the observations that bacteria and archaea utilize CRISPR and the CRISPR-associated (Cas) systems, combined with short RNA to direct degradation of foreign nucleic acids, and applied the concept to gene-editing of human cells. They developed a type II bacterial CRISPR system to function with custom guide RNA (gRNA) in human cells. They used the system to delete the human adeno-associated virus integration site 1 (AAVS1). They obtained targeting rates of 10 to 25% in 293T cells, 13 to 8% in K562 cells, and 2 to 4% in induced pluripotent stem cells [26]. Subsequent variations on the theme were reported, which were effective at deleting human genomic DNA, these methods are incorporated by reference [27, 28].

In some embodiments, a genetically engineered form of (CRISPR)-CRISPR-associated (Cas) protein system [118] of Streptococcus pyogenes is used to induce gene editing of immune checkpoint genes as described for other genes and incorporated by reference [119]. In this system, the type II CRISPR protein Cas9 is directed to genomic target sites by short RNAs, where it functions as an endonuclease. In the naturally occurring system, Cas9 is directed to its DNA target site by two noncoding CRISPR RNAs (crRNAs), including a trans-activating crRNA (tracrRNA) and a precursor crRNA (pre-crRNA). In the synthetically reconstituted system, these two short RNAs can be fused into a single chimeric guide RNA (gRNA). A Cas9 mutant with undetectable endonuclease activity (dCas9) has been targeted to genes in bacteria, yeast, and human cells by gRNAs to silence gene expression through steric hindrance [120].

In some embodiments, disclosed is the use of a regulatory element that is operably linked to one or more elements of a CRISPR system so as to drive expression of the one or more elements of the CRISPR system, with the goal of manipulating DNA encoding for immune checkpoint genes in lymphocytes in a manner that prevents lymphocytes from expressing said checkpoint genes. Immune checkpoint genes relevant for the practice of the invention include : a) the E3 ubiquitin ligase Cbl-b; b) CTLA-4; c) PD-1; d) TIM-3; e) killer inhibitory receptor (KIR); f) LAG-3; g) CD73; h) Fas; i) the aryl hydrocarbon receptor; j) Smad2; k) Smad4; 1) TGF-β receptor; and m) ILT-3.

In the embodiment of the disclose in which an endogenous CRISPR system is utilized to delete immune checkpoint genes, formation of a CRISPR complex (which is made of a guide sequence hybridized to a target sequence and complexed with one or more Cas proteins) will cause cleavage of one or both strands in or near the target sequence. The tracr sequence used for the disclosed methods may comprise or consist of all or a portion of a wild-type tracr sequence, may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. When inducing gene editing in lymphocytes a Cas enzyme, a guide sequence linked to a tracr-mate sequence, and a tracr sequence could each be operably linked to separate regulatory elements on separate vectors. Useful vectors include viral constructs, which are well known in the art, in one preferred embodiment lentiviral constructs are utilized. In one embodiment of the invention, two or more of the elements expressed from the same or different regulatory elements, may be combined in a single vector, with one or more additional vectors providing any components of the CRISPR system not included in the first vector.

In one embodiment of the invention, CRISPR system elements that are combined in a single vector may be arranged in any suitable orientation, such as one element located 5′ with respect to or 3′ with respect to a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence, and a tracr sequence embedded within one or more intron sequences. In some embodiments, the CRISPR enzyme, guide sequence, tracr mate sequence, and tracr sequence are operably linked to and expressed from the same promoter.

In one embodiment of the invention, a vector comprises one or more insertion sites, such as a restriction endonuclease recognition sequence. In some embodiments, one or more insertion sites are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of a CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell.

In one embodiment, gene deletion of immune checkpoint genes is accomplished using a Cas9 nickase that may be used in combination with guide sequence(s), e.g., two guide sequences, which target respectively sense and antisense strands of the DNA target. This combination allows both strands to be nicked and used to induce NHEJ. In some embodiments, an enzyme coding sequence encoding a CRISPR enzyme is codon optimized for expression in lymphocytes. It is known that the predominance of selected tRNAs in a cell is generally a reflection of the codons used most frequently in peptide synthesis. Accordingly, genes can be tailored for optimal gene expression in a given type of lymphocyte based on codon optimization. Codon usage tables are readily available, for example, at the “Codon Usage Database”, and these tables can be adapted in a number of ways [121].

The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art. The guide sequence may be selected to target any target sequence. In some embodiments, the target sequence is a sequence within a genome of a cell. Exemplary target sequences include those that are unique in the target genome. In some embodiments, a guide sequence is selected to reduce the degree of secondary structure within the guide sequence. Secondary structure may be determined by any suitable polynucleotide folding algorithm. Atracr mate sequence includes any sequence that has sufficient complementarity with a tracr sequence to promote one or more of: (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence.

In some embodiments, the disclosed methods comprise the use of RNA interference (e.g., via an RNA interference inducing molecule) for gene silencing or inhibition. As used herein, the terms “RNA interference inducing molecule,” “RNAi molecule,” and “RNAi agent” are used interchangeably herein to refer to an RNA molecule, such as a double stranded or partially double stranded RNA, which functions to inhibit gene expression of a target gene through RNA-mediated target transcript cleavage or RNA interference. Thus, the RNA interference inducing molecule is capable of gene silencing of the target gene when sufficiently provided to a cell expressing the target gene. A double-stranded RNA, such as that used in small interfering RNA (siRNA), has different properties than single-stranded RNA, double-stranded DNA or single-stranded DNA. It is known that an RNA interference inducing molecule does not have to match perfectly to its target sequence. In some embodiments, however, the 5′ and middle part of the antisense (guide) strand of the siRNA is perfectly complementary to the target nucleic acid sequence. In some embodiments, an RNA interference-inducing molecules according to the present disclosure include RNA molecules that have natural or modified nucleotides, natural ribose sugars or modified sugars and natural or modified phosphate backbone. Accordingly, the RNA interference-inducing molecules referred to in the specification include, but are not limited to, unmodified and modified double stranded (ds) RNA molecules including, short-temporal RNA (stRNA), small interfering RNA (siRNA), short-hairpin RNA (shRNA), microRNA (miRNA), double-stranded RNA (dsRNA), (see, e.g. Baulcombe, Science 297:2002-2003, 2002). The dsRNA molecules, e.g. siRNA, also may contain 3′ overhangs, for example 3’UU or 3’TT overhangs. In one embodiment, the siRNA molecules of the present disclosure do not include RNA molecules that comprise ssRNA greater than about 30-40 bases, about 40-50 bases, about 50 bases or more. In one embodiment, the siRNA molecules of the present disclosure comprise a double stranded structure. In one embodiment, the siRNA molecules of the present disclosure are double stranded for more than about 25%, more than about 50%, more than about 60%, more than about 70%, more than about 80%, more than about 90% of their length.

The RNA interference as described to inhibit immunological checkpoint includes RNA molecules having one or more non-natural nucleotides, i.e. nucleotides other than adenine “A”, guanine “G”, uracil “U”, or cytosine “C”, a modified nucleotide residue or a derivative or analog of a natural nucleotide are also useful. Any modified residue, derivative or analog may be used to the extent that it does not eliminate or substantially reduce (by at least 50%) RNAi activity of the dsRNA. These forms thus include, but are not limited to, aminoallyl UTP, pseudo-UTP, 5-I-UTP, 5-I-CTP, 5-Br-UTP, alpha-S ATP, alpha-S CTP, alpha-S GTP, alpha-S UTP, 4-thio UTP, 2-thio-CTP, 2’NH.sub.2 UTP, 2’NH.sub.2 CTP, and 2’F UTP. Such modified nucleotides include, but are not limited to, aminoallyl uridine, pseudo-uridine, 5-I-uridine, 5-I-cytidine, 5-Br-uridine, alpha-S adenosine, alpha-S cytidine, alpha-S guanosine, alpha-S uridine, 4-thio uridine, 2-thio-cytidine, 2’NH₂ uridine, 2’NH₂ cytidine, and 2′ F uridine, including the free pho (NTP) RNA molecules as well as all other useful forms of the nucleotides. The RNA interference as referred herein additionally includes RNA molecules which contain modifications in the ribose sugars, as well as modifications in the “phosphate backbone” of the nucleotide chain. For example, siRNA or miRNA molecules containing α-D-arabinofuranosyl structures in place of the naturally-occurring α-D-ribonucleosides found in RNA can be used in RNA interference according to the present invention (U.S. Pat. No. 5,177,196). Other examples include RNA molecules containing the o-linkage between the sugar and the heterocyclic base of the nucleoside, which confers nuclease resistance and tight complementary strand binding to the oligonucleotides molecules similar to the oligonucleotides containing 2′-O-methyl ribose, arabinose and particularly α-arabinose (U.S. Pat. No. 5,177,196 which is incorporated herein in its entirety by reference). Also, phosphorothioate linkages can be used to stabilize the siRNA and miRNA molecules (U.S. Pat. No. 5,177,196). siRNA and miRNA molecules having various “tails” covalently attached to either their 3′- or to their 5′-ends, or to both, are also been known in the art and can be used to stabilize the siRNA and miRNA molecules delivered using the methods of the present invention. Generally speaking, intercalating groups, various kinds of reporter groups and lipophilic groups attached to the 3′ or 5′ ends of the RNA molecules are well known to one skilled in the art and are useful according to the methods of the present invention. Descriptions of syntheses of 3′-cholesterol or 3′-acridine modified oligonucleotides applicable to preparation of modified RNA molecules useful according to the present invention can be found, for example, in the articles: Gamper, H. B., Reed, M. W., Cox, T., Virosco, J. S., Adams, A. D., Gall, A., Scholler, J. K., and Meyer, R. B. (1993) Facile Preparation and Exonuclease Stability of 3′-Modified Oligodeoxynucleotides. Nucleic Acids Res. 21 145-150; and Reed, M. W., Adams, A. D., Nelson, J. S., and Meyer, R. B., Jr. (1991) Acridine and Cholesterol-Derivatized Solid Supports for Improved Synthesis of 3′-Modified Oligonucleotides. Bioconjugate Chem. 2 217-225 (1993).

Various specific siRNA and miRNA molecules have been described and additional molecules can be designed by one skilled in the art. For example, the miRNA Database at world-wide-web address: sanger.ac.uk, followed by /Software/Rfam/mirna/index provides a useful source to identify additional miRNAs useful according to the present invention (Griffiths-Jones S. NAR, 2004, 32, Database Issue, D109-D111; Ambros V, Bartel B, Bartel D P, Burge C B, Carrington J C, Chen X, Dreyfuss G, Eddy S R, Griffiths-Jones S, Marshall M, Matzke M, Ruvkun G, Tuschl T. RNA, 2003, 9(3), 277-279).

siRNAs also include small hairpin (also called stem loop) RNAs (shRNAs). In one embodiment, these shRNAs are composed of a short, e.g. 19 to 25 nucleotide, antisense strand, followed by a nucleotide loop of 5 to 9 nucleotides, and the analogous sense strand. Alternatively, the sense strand may precede the nucleotide loop structure and the antisense strand may follow.

In another embodiment, siRNAs useful according the methods of the present disclosure are found in WO 05/042719, WO 05/013886, WO 04/039957, and U.S. Pat. App. No. 20040248296 which are incorporated in their entirety herein by reference. Other useful siRNAs useful in the methods of the present invention include, but are not limited to, those found in U.S. Pat. App. Nos. 20050176666, 20050176665, 20050176664, 20050176663, 20050176025, 20050176024, 20050171040, 20050171039, 20050164970, 20050164968, 20050164967, 20050164966, 20050164224, 20050159382, 20050159381, 20050159380, 20050159379, 20050159378, 20050159376, 20050158735, 20050153916, 20050153915, 20050153914, 20050148530, 20050143333, 20050137155, 20050137153, 20050137151, 20050136436, 20050130181, 20050124569, 20050124568, 20050124567, 20050124566, 20050119212, 20050106726, 20050096284, 20050080031, 20050079610, 20050075306, 20050075304, 20050070497, 20050054598, 20050054596, 20050053583, 20050048529, 20040248174, 20050043266, 20050043257, 20050042646, 20040242518, 20040241854, 20040235775, 20040220129, 20040220128, 20040219671, 20040209832, 20040209831, 20040198682, 20040191905, 20040180357, 20040152651, 20040138163, 20040121353, 20040102389, 20040077574, 20040019001, 20040018176, 20040009946, 20040006035, 20030206887, 20030190635, 20030175950, 20030170891, 20030148507, 20030143732, and WO 05/060721, WO 05/060721, WO 05/045039, WO 05/059134, WO 05/045041, WO 05/045040, WO 05/045039, WO 05/027980, WO 05/014837, WO 05/002594, WO 04/085645, WO 04/078181, WO 04/076623, and WO 04/04635, which are all incorporated herein in their entirety by reference.

The RNA interference according to the present invention can be produced using any known techniques such as direct chemical synthesis, for example through processing of longer double stranded RNAs by exposure to recombinant Dicer protein or Drosophila embryo lysates, through an in vitro system derived from S2 cells, using phage RNA polymerase, RNA-dependant RNA polymerase, and DNA based vectors. Use of cell lysates or in vitro processing may further involve the subsequent isolation of the short, for example, 21-23 nucleotide, siRNAs from the lysate. Chemical synthesis may comprise making two single stranded RNA-oligomers followed by the annealing of the two single stranded oligomers into a double stranded RNA. Other examples include methods disclosed in WO 99/32619 and WO 01/68836 that teach chemical and enzymatic synthesis of siRNA. Moreover, numerous commercial services are available for designing and manufacturing specific siRNAs (see, e.g., QIAGEN Inc., Valencia, Calif. and AMBION Inc., Austin, Tex.)

The RNA interference methods useful in the methods of the present disclosure include siRNAs that target gene expression of any protein encoded inside a eukaryotic cell. Examples of these proteins include endogenous mammalian proteins, parasitic proteins, viral proteins encoded by an eukaryotic cell after entry of a virus into the cell. Examples of methods of preparing such RNA interference are shown, for example in an international patent application Nos. PCT/US03/34424 and PCT/US03/34686, the contents and references of all of these patent applications are herein incorporated by reference in their entirety.

Unlike the siRNA delivery methods described in the prior art, the method of the present invention allows targeting of specific cells to minimize or to avoid completely undesired potential side effects of siRNA therapy.

Aspects of the present disclosure are directed to methods for generating tumor associated fibroblast cytotoxic lymphoid cells such as natural killer (NK) cells. Without wishing to be bound by theory, it is believed that NK cells do not attack normal cells expressing MHC class I molecules but mainly attack cells in which the expression of the MHC class I molecules is reduced or lost. In some embodiments, the use of NK cells to kill cancer associated fibroblasts is disclosed. In some embodiments, allogeneic NK cells are used in cell therapy of a cancer or an infectious disease, thus there is advantageously no need to immunize the NK cells to induce them to recognize target cells, therefore an adverse reaction of GVH (Graft-versus-host) disease can be avoided.

In general, the number of cells collectable from a donor by lymphocyte apheresis is limited, and hence, NK cells in number sufficient for killing target cells, such as cancer cells or cells infected with a pathogen, cannot be induced to stay in the body of a recipient until the target cells are killed. In order to retain NK cells in a number sufficient for killing target cells, such as cancer cells or cells infected with a pathogen, it is necessary to frequently repeat the NK cell transplantation, which is a great burden to the patient. Accordingly, disclosed herein is a technique in which NK cells obtained from a donor are once cultured in a test tube to obtain NK cells in number sufficient for killing target cells has been developed. In this technique, use of a serum or feeder cells of an animal is not preferred.

NK cells may be obtained from umbilical cord blood or derived from hematopoetic precursor cells of umbilical cord blood. Umbilical cord blood of various blood types is classified and stored in an umbilical cord blood bank, and thus if umbilical cord blood is used as an origin of NK cells for use in a treatment, it is easy to select, based on the blood type of a patient, an umbilical cord blood of a blood type that has high histocompatibility and low possibility of an adverse reaction caused by the transplantation. Therefore, a technique in which NK cells for use in a treatment are prepared from an umbilical cord blood without using a serum or feeder cells of an animal has recently attract attention. NK cells can be prepared in a number ten thousand times or more in 6 weeks from CD34-positive cells derived from a cryopreserved umbilical cord blood. It cannot be said, however, that the cytotoxic activity of the NK cells obtained by such a method is high. In addition, in the conventional technique for the preparation of NK cells from hematopoietic precursor cells, it is necessary to replace, during the preparation, a medium with one having a different cytokine composition. The present disclosure provides methods for overcoming these deficiencies by either gene silencing or gene editing checkpoint inhibitors on cord blood derived cells that are programmed in vitro to become cytotoxic to tumor cells.

Cord blood is a unique source of starting material for the practice of methods of the disclosure. Various publications describe the use of cord blood in oncology as a substitute for bone marrow [16-21]. Outside the area of oncology, the clinical use of cord blood has expanded into various areas that range from reconstituting a defective immune system [22], to correcting congenital hematological abnormalities [23], to inducing angiogenesis [24]. Cord blood is known to contain a variety of cellular types. In some embodiments of the disclosed methods, it is important to initiate cultures with cells of lymphoid origin, such as NK cells, T cells, or progenitors thereof. In some embodiments, other cells in cord blood may be utilized in order to allow for increased proliferation of T cells in cord blood. In other embodiments, T cells are generated form the cord blood stem cells in vitro. The original clinically attractive feature of cord blood was the high concentration of hematopoietic stem cells, which is similar to that found in bone marrow: approximately 0.1-0.8 CD34+ cells per 100 nucleated cells. However, in contrast to marrow, CD34+ cells from cord blood possess higher proliferative potential in vitro [29], superior numbers of long term culture initiating cells and SCID repopulating cells [30, 31], as well as higher telomerase activity [32]. The potent hematopoietic activity of cord blood derived CD34+ cells may be attributed to the fact that cord blood is a much more developmentally immature source of stem cells as opposed to stem cells derived from adult sources. Attesting to the robust hematopoietic activity of cord blood derived CD34+ cells in comparison to bone marrow cells is the fact that successful reconstitution, albeit delayed, of post-ablative hematopoiesis occurs in patients receiving approximately one tenth of the total nucleated cell number in a cord blood graft compared to a bone marrow graft. The use of cord blood derived hematopoietic stem cells to generate T cells or NK cells in vitro is part of the current disclosure, subsequent or prior to gene silencing and/gene editing for inhibition/deletion of an immune checkpoint protein.

In addition to being a source of hematopoietic cells, cord blood contains potent angiogenesis stimulating cells. Several phenotypes have been ascribed to cord blood angiogenic stimulating cells. In one report, the CD34+, CD1b+ fraction, which is numerically approximately less than half of the CD34+ fraction of cord blood, was demonstrated to possess ability to differentiate into functional endothelial cells in vitro and in vivo [33]. In another report, VEGF-R3+, CD34+ cells were shown to possess not only the ability to differentiate into endothelial cells in vivo, but also to be able to expand on a per cell number by approximately 40-fold in vitro and still maintain function when transferred in vivo. The same study demonstrated that the concentration of this endothelial progenitor fraction found in cord blood CD34+ cells is approximately tenfold higher as compared to bone marrow CD34+ cells [34]. Regardless of the phenotype of the cord blood cell with angiogenesis stimulating ability, unfractionated cord blood mononuclear cells have also been used in numerous animal models [35-37], as well as in the clinic [24], for successful stimulation of angiogenesis. One particularly interesting characteristic of cord blood endothelial progenitors is that they respond by proliferating and stimulating angiogenesis to agents, which normally would inhibit angiogenesis of bone marrow progenitors [37]. In one aspect of the disclosure, these endothelial cells may be transfected with T cell and/or NK cell stimulatory molecules in order to allow for more effective expansion of cord blood lymphoid cells. Without wishing to be bound by theory, in addition to endothelial progenitors, mesenchymal stem cells (described further elsewhere herein), which are found in cord blood, are known to secrete numerous cytokines and growth factors such as VEGF and FGF-2 [38, 39] which stimulate angiogenic processes. In fact, there are reports of mesenchymal stem cells contributing to angiogenesis through direct differentiation into endothelial cells [40].

Aspects of the disclosure comprise use of mesenchymal stem cells. Mesenchymal stem cells are a type of cell capable of differentiating into various non-hematopoietic tissues. Mesenchymal stem cells are classically defined as adhere to plastic and expressing a non-hematopoietic cell surface phenotype, consisting of CD34-, CD45-, HLA-DR-, while possessing markers such as STRO-1, VCAM, CD13, CD29, CD44, CD90, CD105, SH-3, and STRO-1 [41]. To date mesenchymal stem cells have been purified from bone marrow [42], adipose tissue [43], placenta [44, 45], scalp tissue [46] and cord blood [47]. Cord blood-derived mesenchymal stem cells have demonstrated ability to differentiate into a wide variety of tissues in vitro including neuronal [48-50], hepatic [51, 52], osteoblastic [53], and cardiac [47]. An important aspect of this cell population is their anti-inflammatory and immunomodulatory activity. For example, they constitutively secrete immune inhibitory cytokines such as IL-10 and TGF-βwhile maintaining ability to present antigens to T cells, thus suggesting they may act as a tolerogenic antigen presenting cell [54, 55]. Conceptually, the mesenchymal content of umbilical cord blood grafts may explain the tolerogenic capabilities, which some have speculated to be donor specific. Although the majority of published studies have examined bone marrow derived mesenchymal stem cells, and thus are outside the scope of the present review, it is important to note differences between mesenchymal stem cells derived from different sources. A recent study compared mesenchymal stem cells from bone marrow, cord blood and adipose. Cord blood mesenchymal stem cells which were capable of expansion to approximately 20 times, whereas adipose derived cells expanded an average of 8 times and bone marrow derived cells expanded 5 times [56]. This, and other studies support the important role of mesenchymal stem cell content in the biological activities of the cord blood graft. The mesenchymal stem cell component of cord blood may inhibit T cell activation, thus, for use in the methods of the present disclosure (e.g., targeting cancer associated fibroblasts), these cells may need to be themselves either gene silencing/gene edited, or modified to reduce expression of immune suppressive cytokines while enhancing expression of immune stimulatory cytokines. Another possibility is removing these cells from culture.

Cells with markers and activities resembling embryonic stem cells have been found in cord blood. Zhao et al. identified a population of CD34- cells expressing OCT-4, Nanog, SSEA-3 and SSEA-4, which could differentiate into cells of the mesoderm, ectoderm and endoderm lineage. In vivo administration of these cells into the streptozotocin-induced murine model of diabetes was able to significantly reduce hypoglycemia [57]. The existence of cells with such pluripotency in cord blood was also observed by Kogler et al who identified an Unrestricted Somatic Stem Cell (USSC) with capability of differentiation into functional osteoblasts, chondroblasts, adipocytes, hematopoietic and neural cells. USSC were demonstrated to be capable of > 40 population doublings in vitro without spontaneous differentiation or loss of telomere length. Interestingly, administration of these cells (derived from human cord blood) into fetal sheep resulted significant human hematopoiesis (up to 5%), hepatic chimerism with > 20% albumin-producing human parenchymal hepatic cells, as well as detection of human cardiomyocytes. The mechanism of differentiation was not associated with fusion [58]. Support for presence of such pluripotency in cord blood cells also comes from a similar experiment in which CD34+ Lineage- cells were transfected with GFP and administered in utero to goats. GFP+ cells were detected in blood, bone marrow, spleen, liver, kidney, muscle, lung, and heart of the recipient goats (1.2-36% of all cells examined) [59].

When cord blood is directly expanded for T cells or NK cells for the practice of methods of the disclosure, attention needs to be paid to other cells in cord blood that modulate allogenicity. It is not accurate to simply state that cord blood contains the same percentages of cells as found in adult blood but at a more immature and non-immunogeneic state: cord blood cells possess dominant and passive tolerogeneic mechanisms, in part for protecting the infant from excess inflammatory stimuli which would have disastrous consequences on developmental processes.

The most potent antigen presenting cell of the immune system, the dendritic cell, possesses unique properties when it is isolated from cord blood. While circulating dendritic cells from adult blood are potent stimulators of the mixed lymphocyte reaction (MLR), and co-stimulators of mitogen induced T cell proliferation, dendritic cells derived from cord blood are poor, or even inhibitory, to both measures of immune functions [60, 61] [62]. One possible explanation may be that cord blood dendritic cells possess a predominantly lymphoid phenotype and have lower expression of costimulatory molecules as opposed to adult blood-derived dendritic cells [63-66].

Mechanistically, several studies have shown that cord blood dendritic cells are involved in the anti-inflammatory Th2 bias of the neonate [63-65]. Unique properties of immune suppression, or deviation are observed in that cord blood dendritic cell progenitors exhibit enhanced susceptibility both natural and artificial immune suppressants [67]. In support of such non-immune activating properties, when cord blood versus peripheral blood derived dendritic cells are assessed for ability to stimulate immune response to apoptotic or necrotic cells, peripheral blood derived dendritic cells upregulate costimulatory molecules and stimulate T cell proliferation, whereas cord blood derived dendritic cells do not [68]. Given the above-mentioned properties of cord blood cells it conceptually is possible that these cells have an increased predisposition towards tolerogenicity. An example of this is that growth of cord blood progenitors, but not adult, in M-CSF gives rise to a cell population that exhibits potently suppressive tolerogenic dendritic cell phenotype. These cells are not only are poor allostimulators, but are able to expand CD4+ CD25+ T regulatory cells that are capable of inhibiting mixed lymphocyte reactions [69]. Another interesting tolerogenic feature of cord blood dendritic cells is their propensity to secrete large numbers of MHC II-bearing exosomes that lack expression of costimulatory molecules [70]. This type of exosome was used for prevention of autoimmune disease by other authors [71]. Given the immaturity and anti-inflammatory activity of cord blood dendritic cells, it is suggested that cord blood in general will be more poorly immunogenic as compared to other sources of nucleated cells. A comparison may be made between cord blood grafts and liver transplants in that HLA-matching for liver transplants does not seem to effect graft survival [72]. Indeed dendritic cell populations with a primarily lymphoid phenotype, similar to those found in cord blood are known to predominate in the liver [72]. Tissue transplantation across allogeneic barriers is limited by recipient recognition of antigen presenting cells in the graft, especially of dendritic cells, and subsequent launching of immune mediated rejection. Studies in which donor dendritic cell content is depleted or inactivated has allowed for increased survival of allografts, and even induction of tolerance. Given that the dendritic cells in cord blood are not immunogeneic, and actually possess features of tolerogeneicity, it may be possible that transplantation of cord blood Therefore, in some embodiments, a cord blood graft may be manipulated with various immune modulating agents to enhance its tolerogeneicity.

Cord blood has approximately similar concentrations of CD34+ cells compared to bone marrow on a percentage of nucleated cell basis, however, these cells are significantly more active in terms of stimulating hematopoiesis as previously discussed. Disclosed herein are some of the tolerogenic properties of bone marrow CD34+ cells as may be of relevance to those found in cord blood. On the one hand, it is known that hematopoietic progenitor cells are inherently weak immunogens, as witnessed by their poor stimulatory activity of MLR. On the other hand, early studies suggested that CD34+ hematopoietic cells are actually dominantly immune suppressive, in part through elaborating soluble immune inhibitor factors, including, but not limited to TGF-b. There are even publications stating that CD34+ cells possess a “natural suppressor” phenotype and contribute to tumor growth through inhibition of anti-tumor immunity. In accordance with the notion that CD34+ cells may be tolerogenic, these cells have the ability to functionally inactivate immune cells that recognize them. This “veto effect” has been previously suggested as one of the reasons why high dose bone marrow transplants are associated with enhanced engraftment [73, 74]. Supporting this concept are studies in which induction of clinical transplantation tolerance using donor specific bone marrow has been demonstrated [75]. Mechanistically, in a murine model it was shown that the veto-like effect of donor bone marrow transplantation is dependent on expression of FasL on bone marrow cells [76]. Furthermore, human mixed lymphocyte reaction responder cells can be specifically induced to undergo apoptosis by stimulator, but not third party CD34+ cells obtained from bone marrow [77]. Given that one of the reasons for the veto effect is the hypothesis that the hematopoietic compartment needs to protect itself from immune mediated inflammation, and given that cord blood CD34+ cells are more potent hematopoietically than bone marrow CD34+ cells, it would be natural that the cord blood cells would also have higher veto activity as opposed to bone marrow.

In some embodiments, cord blood is utilized as a source of cytotoxic T cells and/or NK cells. In some embodiments, a solution for suspending or culturing living cells (e.g., NK cells obtained from cord blood) is, for example, a saline, a phosphate buffered saline (PBS), a medium, a serum or the like in general. The solution may contain a carrier pharmaceutically acceptable as a pharmaceutical or a quasi-pharmaceutical in some cases. The NK cells obtained by the disclosed methods (e.g., from cord blood) can be applied to treatment and/or prevention of various diseases having sensitivity to NK cells. Examples of such diseases include, but are not limited to, cancers and tumors such as an oral cancer, a gallbladder cancer, a cholangiocarcinoma, a lung cancer, a liver cancer, a colorectal cancer, a kidney cancer, a bladder cancer and leukemia, and infectious diseases caused by viruses, bacteria and the like. A pharmaceutical composition containing NK cells as disclosed herein may contain, in addition to the NK cells, an NK cell precursor, T cells, NKT cells, hematopoietic precursor cells and other cells in some cases. The cell therapy of the present invention may be practiced singly or in combination with surgical treatment, chemotherapy, radiation therapy or the like. In the cell therapies of the present disclosure, the NK cells expanded by the disclosed methods may be transplanted into a patient together with T cells and NKT cells. The NK cells may be transplanted by, for example, intravenous, intraarterial, subcutaneous or intraperitoneal administration in some cases. In the method for preparing NK cells of the present invention, any of media such as, but not limited to, a KBM501 medium (Kohjin Bio Co., Ltd.), a CellGro SCGM medium (registered trademark, Cellgenix, Iwai Chemicals Company), a STEMLINE II (Sigma-Aldrich Co. LLC.), an X-VIVO15 medium (Lonza, Takara Bio Inc.), IMDM, MEM, DMEM and RPMI-1640 may be singly used as or blended in an appropriate ratio to be used as a medium for culturing cells in some cases. The media for culturing cells may be used with supplementation of at least one additional component, for example a serum, a serum albumin, an appropriate protein, a cytokine, or an antibody.

Medium for culturing cell sof the present disclosure (e.g., cord blood cells, hematopoetic precursor cells, NK cells, cytotoxic T cells, cancer associated fibroblasts) may be supplemented with an autologous serum of a subject, a human AB-type serum available from BioWhittaker Inc. or the like, or a donated human serum albumin available from Japanese Red Cross Society in some cases. The autologous serum and the human AB-type serum may be supplemented in a concentration of 1 to 10%, and the donated human serum albumin may be supplemented in a concentration of 1 to 10%. The subject may be a healthy person, or a patient having any of various diseases sensitive to NK cells. The medium may be supplemented with an appropriate protein, a cytokine, an antibody, a compound or another component as long as the effect of expanding NK cells is not impaired. The cytokine may be interleukin 2 (IL-2), interleukin 3 (IL-3), interleukin 7 (IL-7), interleukin 12 (IL-12), interleukin 15 (IL-15), interleukin 21 (IL-21), stem cell factor (SCF), thrombopoietin (TPO) or FMS-like tyrosine kinase 3 ligand (F1t3L). In some embodiments, the IL-2, IL-3, IL-7, IL-12, IL-15, IL-21, SCF, TPO and Flt3L have a human amino acid sequence and are produced by a recombinant DNA technology. The IL-15 may be used at a concentration of 0.1 to 100 ng/mL, such as 20 to 30 ng/mL, or about 25 ng/mL. The SCF may be used at a concentration of 2 to 100 ng/mL, such as 20 to 30 ng/mL, or about 25 ng/mL. The IL-7 may be used at a concentration of 0.5 to 100 ng/mL, such as 20 to 30 ng/mL, or about 25 ng/mL. The F1t3L may be used at a concentration of 1 to 100 ng/mL, such as 20 to 30 ng/mL, or about 25 ng/mL. The TPO may be used at a concentration of 1 to 100 ng/mL, such as 20 to 30 ng/mL, or about 25 ng/mL. Herein, the concentration of the IL-2 may be shown in Japanese Reference Unit (JRU) and International Unit (IU). Since 1 IU corresponds to approximately 0.622 JRU, 1750 JRU/mL corresponds to approximately 2813 IU/mL. The IL-2 may be used at a concentration of 100 to 2900 IU/mL, such as 100 to 2813 IU/mL, or about 2813 IU/mL. In the preparation methods of the present disclosure and in the cell therapy of the present disclosure, in the step of expanding hematopoietic precursor cells, the cells may be cultured in a medium supplemented with IL-15, SCF, IL-7 and Flt3L. The medium may be supplemented further with TPO in some cases. The medium may be replaced at any time after starting the cultivation as long as a desired number of NK cells can be obtained, for example every 3 to 5 days. In some embodiments of the expansion of the hematopoietic precursor cells, the cell growth rate is abruptly lowered in about 5 weeks. Therefore, the expansion of the hematopoietic precursor cells is conducted for about 5 weeks, for example, for 32, 33, 34, 35, 36, 37 or 38 days, after starting the cultivation. Thereafter, from the expanded hematopoietic precursor cells, NK cells are differentiation induced. In the step of differentially inducing NK cells, the cells are cultured in a medium supplemented with IL-2. In some embodiments, the Nk cells are cultured in a medium supplemented with a histone deacetylase inhibitor. In some embodiments, the histone deacetylase inhibitor is valproic acid. NK cells of the disclosure may be cultured in between 0.5 and 5 µM of valproic acid. In some embodiments, NK cells are cultured in about 2 µM valproic acid.

The differentiation induction of NK cells may conducted for between 4 and 10 days, for example, for 5, 6, 7, 8 or 9 days. Here, cultivation conducted for n days under a given culturing condition refers to the cultivation being conducted from a cultivation start date to n days after under the culturing condition, and means that transition to a next culturing condition or cell collection is performed n days after starting the cultivation. In the present invention, the hematopoietic precursor cells may be frozen during the expansion or after completing the expansion, and thawed in accordance with a time of transplantation into a patient to be used for the transplantation into the patient in some cases. The cells may be frozen and thawed by any of methods known to those skilled in the art. For freezing the cells, any of commercially available cryopreservation solutions is used in some cases.

In the expansion methods of the present disclosure, the culture vessel can be, but is not limited to, commercially available dishes, flasks, plates and multi-well plates. The culturing condition is not especially limited as long as the effect of expanding NK cells is not impaired, but a culturing condition of 37° C., 5% CO₂ and a saturated water vapor atmosphere may be employed. Since the purpose of the present disclosure, in some embodiments, is to prepare a large amount of NK cells, it is advantageous that the time period of culturing the cells in the medium is longer because a larger amount of NK cells can be thus obtained. The culture period is not especially limited as long as the NK cells can be expanded to a desired number of cells.

The method and the production of the pharmaceutical compositions of the present disclosure may be practiced under conditions complying with good manufacturing practices (GMP) for pharmaceuticals and quasi-pharmaceuticals. The cytotoxic activity of the NK cells thus prepared is evaluated by a method known to those skilled in the art. In general, the cytotoxic activity may be quantitatively determined by incubating the NK cells (effector cells) and target cells labeled with a radioactive substance, a fluorescent dye or the like, and then measuring a radiation dose or a fluorescence intensity. The target cells may be K562 cells, acute myelogenous leukemia cells, or chronic myelogenous leukemia cells in some cases, but are not limited to these. The properties of the expanded NK cells may be checked by employing RT-PCR, solid phase hybridization, ELISA, Western blotting, immune precipitation, immunonephelometry, FACS, flow cytometry or the like.

Aspects of the present disclosure are directed to cell therapy. The cell therapy of the present invention may comprise a step of expanding hematopoietic precursor cells under a single culturing condition and a step of differentially inducing the cells obtained in the expanding step into NK cells. In the cell therapy, a medium used in the step of expanding hematopoietic precursor cells under a single culturing condition may be supplemented with IL-15, SCF, IL-7 and Flt3L in some cases. In the cell therapy, the medium used in the step of expanding hematopoietic precursor cells under a single culturing condition may be supplemented further with TPO in some cases. In the cell therapy, the step of differentially inducing the NK cells may include culturing the expanded hematopoietic precursor cells under a culturing condition containing IL-2 in some cases. In the cell therapy, the medium used in each of the steps may be supplemented with a human AB-type serum and/or a human serum albumin. In the cell therapy, the hematopoietic precursor cells may be at least one of hematopoietic precursor cells contained in an umbilical cord blood and/or an adult blood cell tissue, hematopoietic precursor cells differentiation induced from induced pluripotent stem cells, embryonic stem cells and/or adult stem cells, and hematopoietic precursor cells directly converted from differentiated cells. In the cell therapy, the step of transplanting the NK cells into a patient may be a step of transplanting the NK cells together with other cells such as T cells or NKT cells in some cases. The cell therapy of the present invention may be employed for treating and/or preventing an infectious disease and/or a cancer.

In some embodiments, production of NK cells by the present method comprises expanding a population of hematopoietic cells and/or hematopoietic precursor cells. During cell expansion, a plurality of hematopoietic cells within the hematopoietic cell population may differentiate into NK cells. In one embodiment, provided herein is a method of producing a population of activated natural killer (NK) cells, comprising: (a) seeding a population of hematopoietic stem or progenitor cells in a first medium comprising interleukin-15 (IL-15) and, in some cases, one or more of stem cell factor (SCF) and interleukin-7 (IL-7), wherein said IL-15, SCF, and IL-7 are not comprised within an undefined component of said medium, such that the population expands, and a plurality of hematopoietic stem or progenitor cells within said population of hematopoietic stem or progenitor cells differentiate into NK cells during said expanding; and (b) expanding the cells from step (a) in a second medium comprising interleukin-2 (IL-2), to produce a population of activated NK cells. In another embodiment, NK cells provided herein are produced by a two-step process of expansion/differentiation and maturation of NK cells. The first and second steps may comprise culturing the cells in media with a unique combination of cellular factors. In certain embodiments, the process involves (a) culturing and expanding a population of hematopoietic cells in a first medium, wherein a plurality of hematopoietic stem or progenitor cells within the hematopoietic cell population differentiate into NK cells; and (b) expanding the NK cells from step (a) in a second medium, wherein the NK cells are further expanded and differentiated, and wherein the NK cells are maturated (e.g., activated or otherwise possessing cytotoxic activity). In certain embodiments, the method includes no intermediary steps between step (a) and (b), no additional culturing steps prior to step (a), and/or no additional steps (e.g., maturation step) after step (b). In certain embodiments, the methods provided herein comprise a first step of culturing and expanding a population of hematopoietic cells in a first medium, wherein a plurality of hematopoietic stem or progenitor cells within the hematopoietic cell population differentiate into NK cells. In some embodiments, culture of the hematopoietic cells as provided herein results in continuous expansion of the hematopoietic cells and differentiation of NK cells from said hematopoietic cells. In certain embodiments, hematopoietic cells, e.g., stem cells or progenitor cells, used in the methods provided herein are expanded and differentiated in the first step using a feeder layer. In other embodiments, hematopoietic cells, e.g., stem cells or progenitor cells, are expanded and differentiated in the first step without the use of a feeder layer. Feeder cell-independent expansion and differentiation of hematopoietic cells can take place in any container compatible with cell culture and expansion, e.g., flask, tube, beaker, dish, multiwell plate, bag or the like. In a specific embodiment, feeder cell-independent expansion of hematopoietic cells takes place in a bag, e.g., a flexible, gas-permeable fluorocarbon culture bag (for example, from American Fluoroseal). In a specific embodiment, the container in which the hematopoietic cells are expanded is suitable for shipping, e.g., to a site such as a hospital or military zone wherein the expanded NK cells are further expanded and differentiated.

In certain embodiments, hematopoietic cells are expanded and differentiated, e.g., in a continuous fashion, in a first culture medium. In one embodiment, the first culture medium is an animal-component free medium. Exemplary animal component-free media useful in the methods provided herein include, but are not limited to, Basal Medium Eagle (BME), Dulbecco’s Modified Eagle’s Medium (DMEM), Glasgow Minimum Essential Medium (GMEM), Dulbecco’s Modified Eagle’s Medium/Nutrient Mixture F-12 Ham (DMEM/F-12), Minimum Essential Medium (MEM), Iscove’s Modified Dulbecco’s Medium (IMDM), Nutrient Mixture F-10 Ham (Ham’s F-10), Nutrient Mixture F-12 Ham (Ham’s F-12), RPMI-1640 Medium, Williams’ Medium E, STEMSPAN® (Cat. No. Stem Cell Technologies, Vancouver, Canada), Glycostem Basal Growth Medium (GBGM®), AIM-V® medium (Invitrogen), X-VIVO® 10 (Lonza), X-VIVO® 15 (Lonza), OPTMIZER (Invitrogen), STEMSPAN® H3000 (STEMCELL Technologies), CELLGRO COMPLETE® (Mediatech), and any modified variants or combinations thereof.

In some embodiments, the first culture medium comprises one or more medium supplements (e.g., nutrients, cytokines and/or factors). Medium supplements suitable for use in the methods provided herein include, for example without limitation, serum such as human serum AB, fetal bovine serum (FBS) or fetal calf serum (FCS), vitamins, bovine serum albumin (BSA), amino acids (e.g., L-glutamine), fatty acids (e.g., oleic acid, linoleic acid or palmitic acid), insulin (e.g., recombinant human insulin), transferrin (iron saturated human transferrin), β-mercaptoethanol, stem cell factor (SCF), Fms-like-tyrosine kinase 3 ligand (Flt3-L), cytokines such as interleukin-2 (IL-2), interleukin-7 (IL-7), interleukin-15 (IL-15), thrombopoietin (Tpo), heparin, or O-acetyl-carnitine (also referred to as acetylcarnitine, O-acetyl-L-camitine or OAC). In a specific embodiment, the medium used herein comprises human serum AB. In another specific embodiment, the medium used herein comprises FBS. In another specific embodiment, the medium used herein comprises OAC.

In certain embodiments, the first medium does not comprise one or more of, granulocyte colony-stimulating factor (G-CSF), granulocyte/macrophage colony stimulating factor (GM-CSF), interleukin-6 (IL-6), macrophage inflammatory Protein 1 α (MIP1a), or leukemia inhibitory factor (LIF).

Thus, in one aspect, provided herein is a two-step method of producing NK cells, wherein said first step comprises expanding and differentiating a population of hematopoietic cells in a first culture medium in the absence of feeder cells, wherein a plurality of hematopoietic cells within said population of hematopoietic cells differentiate into NK cells during said expanding, and wherein the medium comprises SCF at a concentration of about 1 to about 150 ng/mL, IL-2 at a concentration of about 50 to about 1500 IU/mL, IL-7 at a concentration of about 1 to about 150 ng/mL, IL-15 at a concentration 1 to about 150 ng/mL and heparin at a concentration of about 0.1 to about 30 IU/mL. In some embodiments, the SCF, IL-2, IL-7, IL-15 and heparin are not comprised within an undefined component of said medium (e.g., serum). In certain embodiments, said medium comprises one or more of O-acetyl-carnitine (also referred to as acetylcarnitine, O-acetyl-L-camitine or OAC), or a compound that affects acetyl-CoA cycling in mitodronia, thiazovivin, Y-27632, pyintegrin, Rho kinase (ROCK) inhibitors, caspase inhibitors or other anti-apoptotic compounds/peptides, NOVA-RS (Sheffield Bio-Science) or other small-molecule growth enhancers. In certain embodiments, said medium comprises nicotinamide. In certain embodiments, said medium comprises about 0.5 mM-10 mM OAC. In one embodiment, said medium comprises Stemspan® H3000, and/or DMEM:F12 and about 0.5, 1, 2, 3, 4, 5, 6, 7, 8, 9, or 10 mM OAC. In a specific embodiment of the method, said medium is GBGM@. In another specific embodiment, said medium comprises Stemspan@ H3000 and about 5 mM of OAC. In another specific embodiment, said medium comprises DMEM:F12 and about 5 mM of OAC. The OAC can be added anytime during the culturing methods provided herein. In certain embodiments, said OAC is added to the first medium and/or during the first culturing step. In some embodiments, said OAC is added to the first medium on Day 0, 1, 2, 3, 4, 5, 6, 7, 8, 9, 10, 11, 12, 13, 14, 15, 16, 17, 18, 19, 20, 21 of the culture. In a specific embodiment, said OAC is added to the first medium on Day 7 of the first culturing step. In a more specific embodiment, said OAC is added to the first medium on Day 7 of the culture and is present throughout the first and second culturing steps. In certain embodiments, said OAC is added to the second medium and/or during the second culturing step. In some embodiments, said OAC is added to the second medium on Day 22, 23, 24, 25, 26, 27, 28, 29, 30, 31, 32, 33, 34, 35 of the culture.

In another specific embodiment, said medium is IMDM supplemented with about 5-20% BSA, about 1-10 µg/mL recombinant human insulin, about 10-50 pg/mL iron saturated human transferrin and about 10-50 µM P-mercaptoethanol. In another specific embodiment, said medium does not comprise one or more, or any, of IL-11, IL-3, homeobox-B4 (HoxB4), and/or methylcellulose. In other specific embodiments, said medium comprises SCF at a concentration of about 0.1 to about 500 ng/mL; about 5 to about 100 ng/mL; or about 20 ng/mL. In other specific embodiments, said medium comprises IL-2 at a concentration of about 10 to about 2000 IU/mL; or about 100 to about 500 IU/mL; or about 200 IU/mL. In other specific embodiments, said medium comprises IL-7 at a concentration of about 0.1 to about 500 ng/mL; about 5 to about 100 ng/mL; or about 20 ng/mL. In other specific embodiments, said medium comprises IL-15 at a concentration of about 0.1 to about 500 ng/mL; about 5 to about 100 ng/mL; or about 10 ng/mL. In other specific embodiments, said medium comprises heparin at concentration of about 0.05 to about 100 U/mL; or about 0.5 to about 20 U/ml; or about 1.5 U/mL. In yet other specific embodiment of the method, said medium further comprises Fms-like-tyrosine kinase 3 ligand (Flt-3 L) at a concentration of about 1 to about 150 ng/mL, thrombopoietin (Tpo) at a concentration of about 1 to about 150 ng/mL, or a combination of both. In other specific embodiments, said medium comprises Flt-3 L at a concentration of about 0.1 to about 500 ng/mL; about 5 to about 100 ng/mL; or about 20 ng/mL. In other specific embodiments, said medium comprises Tpo at a concentration of about 0.1 to about 500 ng/mL; about 5 to about 100 ng/mL; or about 20 ng/mL. In a more specific embodiment of the method, the first culture medium is GBGM®, which comprises about 20 ng/mL SCF, about 20 ng/mL IL-7, about 10 ng/mL IL-15. In another more specific embodiment of the method, the first culture medium is GBGM@, which comprises about 20 ng/mL SCF, about 20 ng/mL Flt3-L, about 200 IU/mL IL-2, about 20 ng/mL IL-7, about 10 ng/mL IL-15, about 20 ng/mL Tpo, and about 1.5 U/mL heparin. In another specific embodiment, said first culture medium further comprises 10% human serum (e.g., human serum AB) or fetal serum (e.g., FBS).

In some embodiments, NK cells (e.g., NK cells derived from hematopoietic precursor cells) are utilized as a target cell for gene editing. NK cell expansion methods are widely known in the art, for example, in one methodology NK cells are purified by removing T cells from the cell population, after removal of T cells, the remaining cells are cultured in a medium supplemented with 2500 to 3000 IU/mL of IL-2, and transplanting the NK cells which are amplified from the remaining cells to a patient. The method may comprise a step of removing hematopoietic progenitor cells or other cells from the cell population. In the step of transplanting the NK cells to the patient, the gene edited NK cells may be transplanted together with NK cell progenitors, T cells, NKT cells, hematopoietic progenitor cells or the like. One gene that may be edited is the NK KIR gene. In the method for adoptive immunotherapy of the present disclosure, the step of transplanting the NK cells to the patient may be implemented by a step of administering a pharmaceutical composition of the present invention to the patient.

In the adoptive immunotherapy method of the present invention, the cell population which is comprised of NK cells may be prepared from at least one kind of cell selected from a group consisting of: hematopoietic stem cells derived from any stem cells selected from a group consisting embryonic stem cells, adult stem cells and induced pluripotent stem cells (iPS cells); hematopoietic stem cells derived from umbilical cord blood; hematopoietic stem cells derived from peripheral blood; hematopoietic stem cells derived from bone marrow blood; umbilical cord blood mononuclear cells; and peripheral blood mononuclear cells. The donor of the cell population which is comprised of NK cells may be the recipient, that is, the patient himself or herself, a blood relative of the patient, or a person who is not a blood relative of the patient. The NK cells may be derived from a donor whose major histocompatibility antigen complex (MHC) and killer immunoglobulin-like receptors (KIR) do not match with those of the recipient. The gene editing step may be performed on NK progenitor cells, thus circumventing the need for wide-scale transfection.

In the amplifying step of the invention the cell population which is comprised of NK cells may be prepared using various procedures known to those skilled in the art. For example, to collect mononuclear cells from blood such as umbilical cord blood and peripheral blood, the buoyant density separation technique may be employed. NK cells may be collected with immunomagnetic beads. Furthermore, the NK cells may be isolated and identified using a FACS (fluorescent activated cell sorter) or a flow cytometer, following immunofluorescent staining with specific antibodies against cell surface markers. The NK cells may be prepared by separating and removing cells expressing cell surface antigens CD3 and/or CD34, with immunomagnetic beads comprising, but not limited to, Dynabeads® and CliniMACS® of Miltenyi Biotec GmbH. T cells and/or hematopoietic progenitor cells may be selectively injured or killed using specific binding partners for T cells and/or hematopoietic progenitor cells. The step of removing the T cells from the mononuclear cells may be a step of removing cells of other cell types, such as hematopoietic progenitor cells, B cells and/or NKT cells, together with the T cells. The step of removing the hematopoietic progenitor cells from the mononuclear cells may be a step of removing cells of other cell types, such as T cells, B cells and/or NKT cells, together with the hematopoietic progenitor cells. In the amplifying method of the present disclosure, the mononuclear cells separated from the umbilical cord blood and peripheral blood may be cryopreserved and stored to be thawed in time for transplantation to the patient. Alternatively, the mononuclear cells may be frozen during or after amplification by the method for amplifying the NK cells of the present invention, and thawed in time for transplantation to the patient. Any method known to those skilled in the art may be employed in order to freeze and thaw the blood cells. Any commercially available cryopreservation fluid for cells may be used to freeze the cells.

In some embodiments, disclosed are methods for generating a population of cells with tumoricidal ability that have been gene edited. In one example, 50 ml of peripheral blood is extracted from a cancer patient and peripheral blood monoclear cells (PBMC) are isolated using the Ficoll Method. PBMCs are subsequently resuspended in 10 ml STEM-34 media and allowed to adhere onto a plastic surface for 2-4 hours. The adherent cells are then cultured at 37° C. in STEM-34 media supplemented with 1,000 U/mL granulocyte-monocyte colony-stimulating factor and 500 U/mL IL-4 after non-adherent cells are removed by gentle washing in Hanks Buffered Saline Solution (HBSS). Half of the volume of the GM-CSF and IL-4 supplemented media is changed every other day. Immature dendritic cells (DCs) are harvested on day 7. In one embodiment, said generated DCs are used to stimulate T cell and NK cell tumoricidal activity. Specifically, generated DCs may be further purified from culture through use of flow cytometry sorting or magnetic activated cell sorting (MACS), or may be utilized as a semi-pure population. Gene editing may be performed prior to coculture, during coculture, or after coculture. In some embodiments, gene editing is performed prior to coculture. DCs may be provided to a patient in need of therapy in order to stimulate NK and T cell activity in vivo, or in another embodiment may be incubated in vitro with a population of cells containing T cells and/or NK cells. In one embodiment, DCs are exposed to agents capable of stimulating maturation in vitro. Specific methods for stimulating in vitro maturation include culturing DC or DC containing populations with a toll like receptor agonist. Another means of achieving DC maturation involves exposure of DCs to TNF-α at a concentration of approximately 20 ng/mL. In some embodiments, in order to activate T cells and/or NK cells in vitro, cells are cultured in media containing approximately 1000 IU/ml of interferon gamma. Incubation with interferon gamma may be performed for between a period of 2 hours and a period of 7 days. In some embodiments, incubation is performed for approximately 24 hours, after which T cells and/or NK cells are stimulated via the CD3 and CD28 receptors. One means of accomplishing this is by addition of antibodies capable of activating these receptors. In one embodiment, approximately 2 ug/ml of anti-CD3 antibody is added, together with approximately 1 ug/ml anti-CD28. In order to promote survival of T cells and NK cells, was well as to stimulate proliferation, a T cell/NK mitogen may be used. In one embodiment the cytokine IL-2 is utilized. Specific concentrations of IL-2 useful for the practice of the invention are approximately 500 u/mL IL-2. Media containing IL-2 and antibodies may be changed every 48 hours for approximately 8-14 days. In one particular embodiment DCs are provided to said T cells and/or NK cells in order to endow cytotoxic activity towards tumor cells. In a particular embodiment, inhibitors of caspases are added in the culture so as to reduce rate of apoptosis of T cells and/or NK cells. Generated cells can be administered to a subject intradermally, intramuscularly, subcutaneously, intraperitoneally, intraarterially, intravenously (including a method performed by an indwelling catheter), intratumorally, or into an afferent lymph vessel. Gene editing methods that have utilized transfection of T cells with CRISPR-Cas9 are incorporated by reference [122-126].

In some embodiments, the culture of the cells is performed by starting with purified lymphocyte populations. The step of separating the cell population and cell sub-population containing a T cell can be performed, for example, by fractionation of a mononuclear cell fraction by density gradient centrifugation, or a separation means using the surface marker of the T cell as an index. Subsequently, isolation based on surface markers may be performed. Examples of the surface marker include CD3, CD8 and CD4, and separation methods depending on these surface markers are known in the art. For example, the step can be performed by mixing a carrier such as beads or a culturing container on which an anti-CD8 antibody has been immobilized, with a cell population containing a T cell, and recovering a CD8-positive T cell bound to the carrier. CD8 MicroBeads, Dynabeads M450 CD8, and/or Eligix anti-CD8 mAb coated nickel particles can be suitably used. This is also the same as in implementation using CD4 as an index and, for example, CD4 MicroBeads and/or Dynabeads M-450 CD4 can also be used. In some embodiments of the invention, T regulatory cells are depleted before initiation of the culture. Depletion of T regulatory cells may be performed by negative selection by removing cells that express makers such as neuropilin, CD25, CD4, CTLA4, and membrane bound TGF-β.

The step of culturing the cell population and cell sub-population containing a T cell can be performed by selecting suitable known culturing conditions depending on the cell population. In addition, in the step of stimulating the cell population, various proteins and chemical ingredients, etc., may be added to the medium to perform culturing. For example, cytokines, chemokines or other ingredients may be added to the medium. Herein, the cytokine is not particularly limited as far as it can act on the T cell, and examples thereof include IL-2, IFN-.gamma., transforming growth factor (TGF)-β, IL-15, IL-7, IFN-α, IL-12, CD40L, and IL-27. From the viewpoint of enhancing cellular immunity, particularly suitably, IL-2, IFN-.gamma., or IL-12 is used and, from the viewpoint of improvement in survival of a transferred T cell in vivo, IL-7, IL-15 or IL-21 is suitably used. In addition, the chemokine is not particularly limited as far as it acts on the T cell and exhibits migration activity, and examples thereof include RANTES, CCL21, MIP1α, MIP1β, CCL19, CXCL12, IP-10 and MIG. The stimulation of the cell population can be performed by the presence of a ligand for a molecule present on the surface of the T cell, for example, CD3, CD28, or CD44 and/or an antibody to the molecule. Further, the cell population can be stimulated by contacting with other lymphocytes such as antigen presenting cells (dendritic cell) presenting a target peptide such as a peptide derived from a cancer antigen on the surface of a cell. In addition to assessing cytotoxicity and migration as end points, it is within the scope of the current disclosure to optimize the cellular product based on other means of assessing T cell activity, for example, the function enhancement of the T cell in the method of the present invention can be assessed at a plurality of time points before and after each step using, for example, a cytokine assay, an antigen-specific cell assay (tetramer assay), a proliferation assay, a cytolytic cell assay, or an in vivo delayed hypersensitivity test using a recombinant tumor-associated antigen or an immunogenic fragment or an antigen-derived peptide. Examples of additional methods for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, and an assay which is currently used for measuring a T cell response such as a limiting dilution method. In vivo assessment of the efficacy of the generated cells using the disclosed methods may be assessed in a living body before first administration of the disclosed T cells and/or NK cells with enhanced function, or at various time points after initiation of treatment, using an antigen-specific cell assay, a proliferation assay, a cytolytic cell assay, or an in vivo delayed hypersensitivity test using a recombinant tumor-associated antigen or an immunogenic fragment or an antigen-derived peptide. Examples additional methods for measuring an increase in an immune response include a delayed hypersensitivity test, flow cytometry using a peptide major histocompatibility gene complex tetramer, a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, and an assay which is currently used for measuring a T cell response such as a limiting dilution method.

### III. Hematopoietic Precursor Cells

Aspects of the disclosure comprise the use of hematopoietic precursor cells. The term “hematopoietic precursor cells” as used herein describes cells having the potential to differentiate into any type of blood cells. The hematopoietic precursor cells of the present disclosure include, but are not limited to, hematopoietic stem cells derived from umbilical cord blood, hematopoietic stem cells derived from an adult blood cell tissue such as a bone marrow, hematopoietic precursor cells derived from differentiation of induced pluripotent stem cells, embryonic stem cells and/or adult stem cells, and hematopoietic precursor cells derived from differentation of fibroblasts. The hematopoietic precursor cells of the present disclosure may be CD34-positive cells. The hematopoietic precursor cells of the present disclosure may be prepared, however, by using a marker other than CD34 as long as CD34-positive cells are substantially contained. The hematopoietic precursor cells of the present disclosure may be prepared by any procedures known to those skilled in the art. For example, in collecting mononuclear cells from an umbilical cord blood, specific gravity centrifugation may be employed. Hematopoietic precursor cells present in an umbilical cord blood may be selectively collected from mononuclear cells derived from the umbilical cord blood using immunomagnetic beads on which an antibody to a cell surface marker is immobilized. As the immunomagnetic beads, Dynabeads® manufactured by Dynal and available from Invitrogen, or CliniMACS® manufactured by Miltenyi Biotec may be used, but the immunomagnetic beads are not limited to these. On the immunomagnetic beads, an anti-CD34 antibody may be immobilized and utilized to collect hematopoietic precursor cells. Immunomagnetic beads on which another specifically bonding partner such as an antibody to a cell surface marker different from CD34 is immobilized may be used as long as CD34-positive cells derived from the umbilical cord blood can be collected. In some embodiments, the hematopoietic precursor cells can be isolated/identified by performing immunofluorescent staining with a specific antibody to a cell surface marker and by using a flow cytometer. In the expansion method of the present invention, mononuclear cells separated from an umbilical cord blood may be cryopreserved and thawed in accordance with a time of transplantation to a patient to be used for expanding NK cells in some cases. The cryopreservation and thaw of the cells may be performed any method known to those skilled in the art. For the cryopreservation of the cells, any of commercially available cell cryopreservation solutions is used in some cases. In some embodiments of the invention, cells generated from hematopoietic stem cells are used to kill tumor associated fibroblast cells.

If the hematopoietic precursor cells are differentiation induced from induced pluripotent stem cells, embryonic stem cells and/or adult stem cells, the hematopoietic precursor cells may be differentiation induced from undifferentiated pluripotent stem cells by employing a culturing condition not using feeder cells and a serum, such as one reported by Niwa, A. et al., (PLoS ONE 6(7): e22261 (2011)), in some cases. To be brief, human ES cells or human iPS cells are allowed to form colonies in a serum-free medium for retaining the cells in an undifferentiated state, the medium is replaced with a serum-free medium for differentiation induction supplemented with BMP4, and with this day set as day 0, the cells are cultured up to day 4. On day 4, the medium is replaced with a serum-free medium for differentiation induction supplemented with VEGF and SCF instead of BMP4, and the cells are cultured up to day 6. Thereafter, on day 6, the medium is replaced with a serum-free medium for differentiation induction supplemented with a stem cell factor (SCF), thrombopoietin (TPO), interleukin 3 (IL-3), FMS-like tyrosine kinase 3 ligand (F1t3L), a fusion protein of IL-6 receptor and IL-6 (FP6), and the like. On day 10 to 12, a cluster of hematopoietic cells starts to be observed in a margin of the colony, and starts to float in the medium several days later.

### IV. Cultured Cells

Aspects of the present disclosure comprise cells useful in therapeutic methods and compositions. Cells disclosed herein include, for example, fibroblasts, stem cells (e.g., hematopoietic stem cells or mesenchymal stem cells), and endothelial progenitor cells. Cells of a given type (e.g., fibroblasts) may be used alone or in combination with cells of other types. For example, fibroblasts may be isolated and provided to a subject alone or in combination with one or more stem cells. In some embodiments, fibroblasts of the present disclosure are adherent to plastic. In some embodiments, the fibroblasts express CD73, CD90, and/or CD105. In some embodiments, the fibroblasts are CD14, CD34, CD45, and/or HLA-DR negative. In some embodiments, the fibroblasts possess the ability to differentiate to osteogenic, chondrogenic, and adipogenic lineage cells.

Compositions of the present disclosure may be obtained from isolated fibroblast cells or a population thereof capable of proliferating and differentiating into ectoderm, mesoderm, or endoderm. In some embodiments, an isolated fibroblast cell expresses at least one of Oct-4, Nanog, Sox-2, KLF4, c-Myc, Rex-1, GDF-3, LIF receptor, CD105, CD117, CD344 or Stella markers. In some embodiments, an isolated fibroblast cell does not express at least one of MHC class I, MHC class II, CD45, CD13, CD49c, CD66b, CD73, CD105, or CD90 cell surface proteins. Such isolated fibroblast cells may be used as a source of conditioned media. The cells may be cultured alone, or may by cultured in the presence of other cells in order to further upregulate production of growth factors in the conditioned media.

In some embodiments, fibroblasts of the present disclosure express telomerase, Nanog, Sox2, β-III-Tubulin, NF-M, MAP2, APP, GLUT, NCAM, NeuroD, Nurr1, GFAP, NG2, Olig1, Alkaline Phosphatase, Vimentin, Osteonectin, Osteoprotegrin, Osterix, Adipsin, Erythropoietin, SM22-α, HGF, c-MET, α-1-Antriptrypsin, Ceruloplasmin, AFP, PEPCK 1, BDNF, NT-4/5 TrkA, BMP2, BMP4, FGF2, FGF4, PDGF, PGF, TGFα, TGFβ, and/or VEGF.

Fibroblasts may be expanded and utilized by administration themselves, or may be cultured in a growth media in order to obtain conditioned media. The term Growth Medium generally refers to a medium sufficient for the culturing of fibroblasts. In particular, one presently preferred medium for the culturing of the cells of the invention herein comprises Dulbecco’s Modified Essential Media (DMEM). Particularly preferred is DMEM-low glucose (also DMEM-LG herein) (Invitrogen®, Carlsbad, Calif.). The DMEM-low glucose is preferably supplemented with 15% (v/v) fetal bovine serum (e.g. defined fetal bovine serum, Hyclone®, Logan Utah), antibiotics/antimycotics (preferably penicillin (100 Units/milliliter), streptomycin (100 milligrams/milliliter), and amphotericin B (0.25 micrograms/milliliter), (Invitrogen®, Carlsbad, Calif.)), and 0.001% (v/v) 2-mercaptoethanol (Sigma®, St. Louis Mo.). In some cases different growth media are used, or different supplementations are provided, and these are normally indicated as supplementations to Growth Medium. Also relating to the present invention, the term standard growth conditions, as used herein refers to culturing of cells at 37° C., in a standard atmosphere comprising 5% CO₂, where relative humidity is maintained at about 100%. While the foregoing conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like.

Also disclosed herein are cultured cells. Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number, or the “doubling time”.

Fibroblast cells used in the disclosed methods can undergo at least 25, 30, 35, or 40 doublings prior to reaching a senescent state. Methods for deriving cells capable of doubling to reach 10¹⁴ cells or more are provided. Examples are those methods which derive cells that can double sufficiently to produce at least about 10¹⁴, 10¹⁵, 10¹⁶, or 10¹⁷ or more cells when seeded at from about 10³ to about 10⁶ cells/cm² in culture. Preferably these cell numbers are produced within 80, 70, or 60 days or less. In one embodiment, fibroblast cells used are isolated and expanded, and possess one or more markers selected from a group consisting of CD10, CD13, CD44, CD73, CD90, CD141, PDGFr-alpha, HLA-A, HLA-B, and HLA-C. In some embodiments, the fibroblast cells do not produce one or more of CD31, CD34, CD45, CD117, CD141, HLA-DR, HLA-DP, or HLA-DQ.

When referring to cultured cells, including fibroblast cells and vertebrae cells, the term senescence (also “replicative senescence” or “cellular senescence”) refers to a property attributable to finite cell cultures; namely, their inability to grow beyond a finite number of population doublings (sometimes referred to as Hayflick’s limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are resistant to programmed cell death (apoptosis) and can be maintained in their nondividing state for as long as three years. These cells are alive and metabolically active, but they do not divide.

In some cases, fibroblast cells are obtained from a biopsy, and the donor providing the biopsy may be either the individual to be treated (autologous), or the donor may be different from the individual to be treated (allogeneic). In cases wherein allogeneic fibroblast cells are utilized for an individual, the fibroblast cells may come from one or a plurality of donors.

The fibroblasts may be fibroblasts obtained from various sources including, for example, dermal fibroblasts; placental fibroblasts; adipose fibroblasts; bone marrow fibroblasts; foreskin fibroblasts; umbilical cord fibroblasts; hair follicle derived fibroblasts; nail derived fibroblasts; endometrial derived fibroblasts; keloid derived fibroblasts; and fibroblasts obtained from a plastic surgery-related by-product. In some embodiments, fibroblasts are dermal fibroblasts.

In some embodiments, fibroblasts are manipulated or stimulated to produce one or more factors. In some embodiments, fibroblasts are manipulated or stimulated to produce leukemia inhibitory factor (LIF), brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-y, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β, vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and/or TGFβ-3. Factors from manipulated or stimulated fibroblasts may be present in conditioned media and collected for therapeutic use.

In some embodiments, fibroblasts are transfected with one or more angiogenic genes to enhance ability to promote angiogenesis. An “angiogenic gene” describes a gene encoding for a protein or polypeptide capable of stimulating or enhancing angiogenesis in a culture system, tissue, or organism. Examples of angiogenic genes which may be useful in transfection of fibroblasts include activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation sphingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin, fibronectin receptor, Factor X, HB-EGF, HBNF, HGF, HUAF, heart derived inhibitor of vascular cell proliferation, IL1, IGF-2 IFN-gamma, α1β1 integrin, α2β1 integrin, K-FGF, LIF, leiomyoma-derived growth factor, MCP-1, macrophage-derived growth factor, monocyte-derived growth factor, MD-ECI, MECIF, MMP2, MMP3, MMP9, urokiase plasminogen activator, neuropilin, neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-β, TGF-βreceptors, TIMPs, TNF-α, transferrin, thrombospondin, urokinase, VEGF-A, VEGF-B, VEGF-C, VEGF-D, VEGF-E, VEGF, VEGF(164), VEGI, and EG-VEGF. Fibroblasts transfected with one or more angiogenic factors may be used in the disclosed methods of disease treatment or prevention.

Under appropriate conditions, fibroblasts may be capable of producing interleukin-1 (IL-1) and/or other inflammatory cytokines. In some embodiments, fibroblasts of the present disclosure are modified (e.g., by gene editing) to prevent or reduce expression of IL-1 or other inflammatory cytokines. For example, in some embodiments, fibroblasts are fibroblasts having a deleted or non-functional IL-1 gene, such that the fibroblasts are unable to express IL-1. Such modified fibroblasts may be useful in the therapeutic methods of the present disclosure by having limited pro-inflammatory capabilities when provided to a subject. In some embodiments, fibroblasts are treated with (e.g., cultured with) TNF-α, thereby inducing expression of growth factors and/or fibroblast proliferation.

In some embodiments, fibroblasts of the present disclosure are used as precursor cells that differentiate following introduction into an individual. In some embodiments, fibroblasts are subjected to differentiation into a different cell type (e.g., a hematopoietic cell) prior to introduction into the individual.

As disclosed herein, fibroblasts may secret one or more factors prior to or following introduction into an individual. Such factors include, but are not limited to, growth factors, trophic factors and cytokines. In some instances, the secreted factors can have a therapeutic effect in the individual. In some embodiments, a secreted factor activates the same cell. In some embodiments, the secreted factor activates neighboring and/or distal endogenous cells. In some embodiments, the secreted factor stimulated cell proliferation and/or cell differentiation. In some embodiments, fibroblasts secrete a cytokine or growth factor selected from human growth factor, fibroblast growth factor, nerve growth factor, insulin-like growth factors, hematopoietic stem cell growth factors, a member of the fibroblast growth factor family, a member of the platelet-derived growth factor family, a vascular or endothelial cell growth factor, and a member of the TGFβfamily.

In some embodiments, fibroblasts of the present disclosure are cultured with an inhibitor of mRNA degradation. In some embodiments, fibroblasts are cultured under conditions suitable to support reprogramming of the fibroblasts. In some embodiments, such conditions comprise temperature conditions of between 30° C. and 38° C., between 31° C. and 37° C., or between 32° C. and 36° C. In some embodiments, such conditions comprise glucose at or below 4.6 g/l, 4.5 g/l, 4 g/1, 3 g/1, 2 g/1or 1 g/1. In some embodiments, such conditions comprise glucose of about 1 g/1.

Aspects of the present disclosure comprise generating conditioned media from fibroblasts. Conditioned medium may be obtained from culture with fibroblasts. The cells may be cultured for about 1, 2, 3, 4, 5, 6, 7, 8, 9, 10 days or more. In some embodiments, the fibroblasts are cultured for about 3 days prior to collecting conditioned media. Conditioned media may be obtained by separating the cells from the media. Conditioned media may be centrifuged (e.g., at 500 xg). Conditioned media may be filtered through a membrane. The membrane may be a >1000 kDa membrane. Conditioned media may be subject to liquid chromatography such as HPLC. Conditioned media may be separated by size exclusion.

### V. Administration of Therapeutic Compositions

The therapy provided herein may comprise administration of a therapeutic agents (e.g., fibroblasts, exosomes from fibroblasts, etc.) alone or in combination. Therapies may be administered in any suitable manner known in the art. For example, a first and second treatment may be administered sequentially (at different times) or concurrently (at the same time). In some embodiments, the first and second treatments are administered in a separate composition. In some embodiments, the first and second treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents (e.g., fibroblasts) of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual’s clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 µg/kg, mg/kg, µg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 µM to 150 µM. In another embodiment, the effective dose provides a blood level of about 4 µM to 100 µM.; or about 1 µM to 100 µM; or about 1 µM to 50 µM; or about 1 µM to 40 µM; or about 1 µM to 30 µM; or about 1 µM to 20 µM; or about 1 µM to 10 µM; or about 10 µM to 150 µM; or about 10 µM to 100 µM; or about 10 µM to 50 µM; or about 25 µM to 150 µM; or about 25 µM to 100 µM; or about 25 µM to 50 µM; or about 50 µM to 150 µM; or about 50 µM to 100 µM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 µM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of µg/kg or mg/kg of body weight can be converted and expressed in comparable concentration units of µg/m1or mM (blood levels), such as 4 µM to 100 µM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In some embodiments, between about 10⁵ and about 10¹³ cells per 100 kg are administered to a human per infusion. In some embodiments, between about 1.5x10⁶ and about 1.5x 10¹² cells are infused per 100 kg. In some embodiments, between about 1x10⁹ and about 5x10¹¹ cells are infused per 100 kg. In some embodiments, between about 4x10⁹ and about 2x10¹¹ cells are infused per 100 kg. In some embodiments, between about 5x10⁸ cells and about 1x10¹ cells are infused per 100 kg. In some embodiments, a single administration of cells is provided. In some embodiments, multiple administrations are provided. In some embodiments, multiple administrations are provided over the course of 3-7 consecutive days. In some embodiments, 3-7 administrations are provided over the course of 3-7 consecutive days. In some embodiments, 5 administrations are provided over the course of 5 consecutive days. In some embodiments, a single administration of between about 10⁵ and about 10¹³ cells per 100 kg is provided. In some embodiments, a single administration of between about 1.5x10⁸ and about 1.5x 10¹² cells per 100 kg is provided. In some embodiments, a single administration of between about 1x10⁹ and about 5x10¹¹ cells per 100 kg is provided. In some embodiments, a single administration of about 5x10¹⁰ cells per 100 kg is provided. In some embodiments, a single administration of 1x10¹⁰ cells per 100 kg is provided. In some embodiments, multiple administrations of between about 10⁵ and about 10¹³ cells per 100 kg are provided. In some embodiments, multiple administrations of between about 1.5x10⁸ and about 1.5x 10¹² cells per 100 kg are provided. In some embodiments, multiple administrations of between about 1x10⁹ and about 5x10¹¹ cells per 100 kg are provided over the course of 3-7 consecutive days. In some embodiments, multiple administrations of about 4x10⁹ cells per 100 kg are provided over the course of 3-7 consecutive days. In some embodiments, multiple administrations of about 2x10¹¹ cells per 100 kg are provided over the course of 3-7 consecutive days. In some embodiments, 5 administrations of about 3.5x10⁹ cells are provided over the course of 5 consecutive days. In some embodiments, 5 administrations of about 4x10⁹ cells are provided over the course of 5 consecutive days. In some embodiments, 5 administrations of about 1.3x10¹¹ cells are provided over the course of 5 consecutive days. In some embodiments, 5 administrations of about 2x10¹¹ cells are provided over the course of 5 consecutive days.

### VI. Kits of the Disclosure

Any of the cellular and/or non-cellular compositions described herein or similar thereto may be comprised in a kit. In a non-limiting example, one or more reagents for use in methods for preparing fibroblasts or derivatives thereof (e.g., exosomes derived from fibroblasts) may be comprised in a kit. Such reagents may include cells, vectors, one or more growth factors, vector(s) one or more costimulatory factors, media, enzymes, buffers, nucleotides, salts, primers, compounds, and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### EXAMPLE

The following example is included to demonstrate particular embodiments of the disclosure. It should be appreciated by those of skill in the art that the techniques disclosed in the example that follows represent techniques discovered by the inventors to function well in the practice of the methods of the disclosure, and thus can be considered to constitute particular modes for its practice. However, those of skill in the art should, in light of the present disclosure, appreciate that many changes can be made in the specific embodiments that are disclosed and still obtain a like or similar result without departing from the spirit and scope of the disclosure.

### Example 1: Cord Blood-Derived Natural Killer Cells Selectively Target Cancer Associated Fibroblasts for Killing

Cancer associated fibroblasts where generated by culture of dermal fibroblasts obtained from the American Type Culture Collection (ATCC®) with conditioned media from PC-3 prostate cancer cell line for 2 weeks. Control fibroblasts where cultured in OPTIMEM media, all cultures had 10% fetal calf serum.

Umbilical cord blood cytotoxic cells where generated by exposing cord blood mononuclear cells to interleukin-2 (100 IU/ml) together with anti-CD3/anti-CD28 beads and 2 µM of the histone deacetylase inhibitor valproic acid. After 2 weeks of culture, CD56⁺ Natural Killer (NK) cells where isolated.

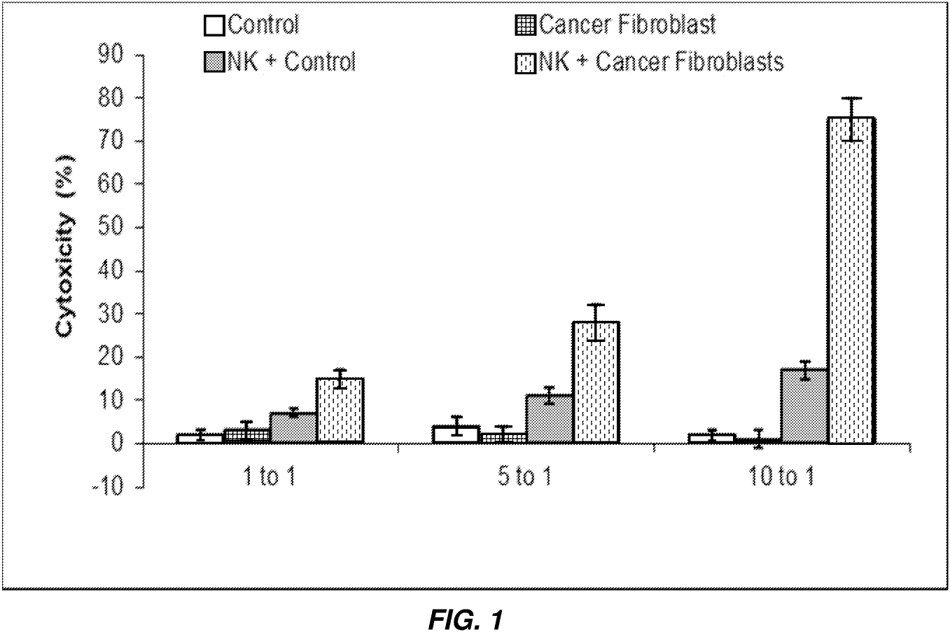

Umbilical cord blood CD56⁺ cells were cultured at the indicated ratios and cytotoxicity was assessed by chromium release. As shown in FIG. 1, cord blood cells selectively killed cancer-associated fibroblasts but not control fibroblasts.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.
1. Bryceson, Y.T. and H.G. Ljunggren, Tumor cell recognition by the NK cell activating receptor NKG2D. Eur J Immunol, 2008. 38(11): p. 2957-61.
2. Waldhauer, I. and A. Steinle, NK cells and cancer immunosurveillance. Oncogene, 2008. 27(45): p. 5932-43.
3. Guerra, N., et al., NKG2D-deficient mice are defective in tumor surveillance in models of spontaneous malignancy. Immunity, 2008. 28(4): p. 571-80.
4. Guillerey, C., et al., Immunosurveillance and therapy of multiple myeloma are CD226 dependent. J Clin Invest, 2015. 125(5): p. 2077-89.
5. Horn, T., et al., The prognostic effect of tumour-infiltrating lymphocytic subpopulations in bladder cancer. World J Urol, 2015.
6. de Jong, R.A., et al., Presence of tumor-infiltrating lymphocytes is an independent prognostic factor in type I and II endometrial cancer. Gynecol Oncol, 2009. 114(1): p. 105-10.
7. Leffers, N., et al., Prognostic significance of tumor-infiltrating T-lymphocytes in primary and metastatic lesions of advanced stage ovarian cancer. Cancer Immunol Immunother, 2009. 58(3): p. 449-59.
8. Coquet, J.M., et al., Epithelial and dendritic cells in the thymic medulla promote CD4+Foxp3+ regulatory T cell development via the CD27-CD70 pathway. J Exp Med, 2013. 210(4): p. 715-28.
9. Cowan, J.E., et al., The thymic medulla is required for Foxp3+ regulatory but not conventional CD4+ thymocyte development. J Exp Med, 2013. 210(4): p. 675-81.
10. Bautista, J.L., et al., Intraclonal competition limits the fate determination of regulatory T cells in the thymus. Nat Immunol, 2009. 10(6): p. 610-7.
11. Ochs, H.D., E. Gambineri, and T.R. Torgerson, IPEX, FOXP3 and regulatory T-cells: a model ƒor autoimmunity. Immunol Res, 2007. 38(1-3): p. 112-21.
12. Jie, H.B., et al., CTLA-4+ Regulatory T Cells Increased in Cetuximab-Treated Head and Neck Cancer Patients Suppress NK Cell Cytotoxicity and Correlate with Poor Prognosis. Cancer Res, 2015. 75(11): p. 2200-10.
13. Hanakawa, H., et al., Regulatory T-cell infiltration in tongue squamous cell carcinoma. Acta Otolaryngol, 2014. 134(8): p. 859-64.
14. Kim, S.T., et al., Tumor-infiltrating lymphocytes, tumor characteristics, and recurrence in patients with early breast cancer. Am J Clin Oncol, 2013. 36(3): p. 224-31.
15. Herbst, R.S., et al., Predictive correlates of response to the anti-PD-L1 antibody MPDL3280A in cancer patients. Nature, 2014. 515(7528): p. 563-7.
16. Cornetta, K., et al., Umbilical cord blood transplantation in adults: results of the prospective Cord Blood Transplantation(COBLT). Biol Blood Marrow Transplant, 2005. 11(2): p. 149-60.
17. Schonberger, S., et al., Transplantation of haematopoietic stem cells derived from cord blood, bone marrow or peripheral blood: a single centre matched-pair analysis in a heterogeneous risk population. Klin Padiatr, 2004. 216(6): p. 356-63.
18. Lekakis, L., et al., Phase II study of unrelated cord blood transplantation for adults with high-risk hematologic malignancies. Bone Marrow Transplant, 2006. 38(6): p. 421-6.
19. Tomonari, A., et al., Cord blood transplantation for acute myelogenous leukemia using a conditioning regimen consisting of granulocyte colony-stimulating factor-combined high-dose cytarabine, fludarabine, and total body irradiation. Eur J Haematol, 2006. 77(1): p. 46-50.
20. Laporte, J.P., et al., Unrelated mismatched cord blood transplantation in patients with hematological malignancies: a single institution experience. Bone Marrow Transplant, 1998. 22 Suppl 1: p. S76-7.
21. Sanz, G.F., et al., Unrelated donor cord blood transplantation in adults with chronic myelogenous leukemia: results in nine patients from a single institution. Bone Marrow Transplant, 2001. 27(7): p. 693-701.
22. Knutsen, A.P. and D.A. Wall, Umbilical cord blood transplantation in severe T-cell immunodeficiency disorders: two-year experience. J Clin Immunol, 2000. 20(6): p. 466-76.
23. Jaing, T.H., et al., Rapid and complete donor chimerism after unrelated mismatched cord blood transplantation in 5 children with beta-thalassemia major. Biol Blood Marrow Transplant, 2005. 11(5): p. 349-53.
24. Tomonari, A., et al., Resolution of Behcet’s disease after HLA-mismatched unrelated cord blood transplantation for myelodysplastic syndrome. Ann Hematol, 2004. 83(7): p. 464-6.
25. Barrangou, R., et al., CRISPR provides acquired resistance against viruses in prokaryotes. Science, 2007. 315(5819): p. 1709-12.
26. Mali, P., et al., RNA-guided human genome engineering via Cas9. Science, 2013. 339(6121): p. 823-6.
27. Cho, S.W., et al., Targeted genome engineering in human cells with the Cas9 RNA-guided endonuclease. Nat Biotechnol, 2013. 31(3): p. 230-2.
28. Wang, H., et al., One-step generation of mice carrying mutations in multiple genes by CRISPR/Cas-mediated genome engineering. Cell, 2013. 153(4): p. 910-8.
29. Theunissen, K. and C.M. Verfaillie, A multifactorial analysis of umbilical cord blood, adult bone marrow and mobilized peripheral blood progenitors using the improved ML-IC assay. Exp Hematol, 2005. 33(2): p. 165-72.
30. Ng, Y.Y., et al., Gene-expression profiling of CD34+ cells from various hematopoietic stem-cell sources reveals functional differences in stem-cell activity. J Leukoc Biol, 2004. 75(2): p. 314-23.
31. Hogan, C.J., et al., Engraftment and development of human CD34(+)-enriched cells from umbilical cord blood in NOD/LtSz-scid/scid mice. Blood, 1997. 90(1): p. 85-96.
32. Sakabe, H., et al., Human cord blood-derived primitive progenitors are enriched in CD34+c-kit- cells: correlation between long-term culture-initiating cells and telomerase expression. Leukemia, 1998. 12(5): p. 728-34.
33. Hildbrand, P., et al., The role of angiopoietins in the development of endothelial cells from cord blood CD34+ progenitors. Blood, 2004. 104(7): p. 2010-9.
34. Salven, P., et al., VEGFR-3 and CD133 identify a population of CD34+ lymphatic/vascular endothelial precursor cells. Blood, 2003. 101(1): p. 168-72.
35. Cho, S.W., et al., Enhancement of Angiogenic Efficacy of Human Cord Blood Cell Transplantation. Tissue Eng, 2006.
36. Botta, R., et al., Heart infarct in NOD-SCID mice: therapeutic vasculogenesis by transplantation of human CD34+ cells and low dose CD34+KDR+ cells. Faseb J, 2004. 18(12): p. 1392-4.
37. Le Ricousse-Roussanne, S., et al., Ex vivo differentiated endothelial and smooth muscle cells from human cord blood progenitors home to the angiogenic tumor vasculature. Cardiovasc Res, 2004. 62(1): p. 176-84.
38. Mayer, H., et al., Vascular endothelial growth factor (VEGF-A) expression in human mesenchymal stem cells: autocrine and paracrine role on osteoblastic and endothelial differentiation. J Cell Biochem, 2005. 95(4): p. 827-39.
39. Liu, C.H. and S.M. Hwang, Cytokine interactions in mesenchymal stem cells from cord blood. Cytokine, 2005. 32(6): p. 270-9.
40. Gang, E.J., et al., In vitro endothelial potential of human UC blood-derived mesenchymal stem cells. Cytotherapy, 2006. 8(3): p. 215-27.
41. De Ugarte, D.A., et al., Differential expression of stem cell mobilization-associated molecules on multi-lineage cells from adipose tissue and bone marrow. Immunol Lett, 2003. 89(2-3): p. 267-70.
42. Vaananen, H.K., Mesenchymal stem cells. Ann Med, 2005. 37(7): p. 469-79.
43. Knippenberg, M., et al., Adipose tissue-derived mesenchymal stem cells acquire bone cell-like responsiveness to fluid shear stress on osteogenic stimulation. Tissue Eng, 2005. 11(11-12): p. 1780-8.
44. Portmann-Lanz, C.B., et al., Placental mesenchymal stem cells as potential autologous graft ƒor pre- and perinatal neuroregeneration. Am J Obstet Gynecol, 2006. 194(3): p. 664-73.
45. Zhang, X., et al., Mesenchymal progenitor cells derived from chorionic villi of human placenta for cartilage tissue engineering. Biochem Biophys Res Commun, 2006. 340(3): p. 944-52.
46. Shih, D.T., et al., Isolation and characterization of neurogenic mesenchymal stem cells in human scalp tissue. Stem Cells, 2005. 23(7): p. 1012-20.
47. Kadivar, M., et al., In vitro cardiomyogenic potential of human umbilical vein-derived mesenchymal stem cells. Biochem Biophys Res Commun, 2006. 340(2): p. 639-47.
48. Fu, Y.S., et al., Conversion of human umbilical cord mesenchymal stem cells in Wharton’sjelly to dopaminergic neurons in vitro: potential therapeutic application for Parkinsonism. Stem Cells, 2006. 24(1): p. 115-24.
49. Tondreau, T., et al., Mesenchymal stem cells derived ƒrom CD133-positive cells in mobilized peripheral blood and cord blood: proliferation, Oct4 expression, and plasticity. Stem Cells, 2005. 23(8): p. 1105-12.
50. Jeong, J.A., et al., Rapid neural differentiation of human cord blood-derived mesenchymal stem cells. Neuroreport, 2004. 15(11): p. 1731-4.
51. Kang, X.Q., et al., Fibroblast growth factor-4 and hepatocyte growth factor induce differentiation of human umbilical cord blood-derived mesenchymal stem cells into hepatocytes. World J Gastroenterol, 2005. 11(47): p. 7461-5.
52. Hong, S.H., et al., In vitro differentiation of human umbilical cord blood-derived mesenchymal stem cells into hepatocyte-like cells. Biochem Biophys Res Commun, 2005. 330(4): p. 1153-61.
53. Hutson, E.L., S. Boyer, and P.G. Genever, Rapid isolation, expansion, and differentiation of osteoprogenitorsƒrom ƒull-term umbilical cord blood. Tissue Eng, 2005. 11(9-10): p. 1407-20.
54. Liu, J., et al., Suppression of human peripheral blood lymphocyte proliferation by immortalized mesenchymal stem cells derived from bone marrow of Banna Minipig inbred-line. Transplant Proc, 2004. 36(10): p. 3272-5.
55. Togel, F., et al., Administered mesenchymal stem cells protect against ischemic acute renal failure through differentiation-independent mechanisms. Am J Physiol Renal Physiol, 2005. 289(1): p. F31-42.
56. Kern, S., et al., Comparative Analysis of Mesenchymal Stem Cells from Bone Marrow, Umbilical Cord Blood or Adipose Tissue. Stem Cells, 2006.
57. Zhao, Y., H. Wang, and T. Mazzone, Identification of stem cells from human umbilical cord blood with embryonic and hematopoietic characteristics. Exp Cell Res, 2006. 312(13): p. 2454-64.
58. Kogler, G., et al., A new human somatic stem cell from placental cord blood with intrinsic pluripotent differentiation potential. J Exp Med, 2004. 200(2): p. 123-35.
59. Zeng, F., et al., Multiorgan engraftment and differentiation of human cord blood CD34+ Lin- cells in goats assessed by gene expression profiling. Proc Natl Acad Sci USA, 2006. 103(20): p. 7801-6.
60. Borras, F.E., et al., Identification of both myeloid CD11c+ and lymphoid CD11c-dendritic cell subsets in cord blood. Br J Haematol, 2001. 113(4): p. 925-31.
61. Kawano, Y., T. Noma, and J. Yata, Analysis of decreased autologous mixed lymphocyte reaction of cord blood lymphocytes: with special reference to production of and response to interleukin-2 (IL-2). Asian Pac J Allergy Immunol, 1984. 2(1): p. 49-55.
62. Petty, R.E. and D.W. Hunt, Neonatal dendritic cells. Vaccine, 1998. 16(14-15): p. 1378-82.
63. Sorg, R.V., G. Kogler, and P. Wernet, Identification oƒ cord blood dendritic cells as an immature CD11c- population. Blood, 1999. 93(7): p. 2302-7.
64. Han, P., T. McDonald, and G. Hodge, Potential immaturity of the T-cell and antigen-presenting cell interaction in cord blood with particular emphasis on the CD40-CD40 ligand costimulatory pathway. Immunology, 2004. 113(1): p. 26-34.
65. Drohan, L., et al., Selective developmental defects oƒ cord blood antigen-presenting cell subsets. Hum Immunol, 2004. 65(11): p. 1356-69.
66. De Wit, D., et al., Impaired responses to toll-like receptor 4 and toll-like receptor 3 ligands in human cord blood. J Autoimmun, 2003. 21(3): p. 277-81.
67. Mainali, E.S. and J.G. Tew, Dexamethasone selectively inhibits differentiation oƒ cord blood stem cell derived-dendritic cell (DC) precursors into immature DCs. Cell Immunol, 2004. 232(1-2): p. 127-36.
68. Wong, O.H., F.P. Huang, and A.K. Chiang, Differential responses of cord and adult blood-derived dendritic cells to dying cells. Immunology, 2005. 116(1): p. 13-20.
69. Li, G., Y.J. Kim, and H.E. Broxmeyer, Macrophage colony-stimulating factor drives cord blood monocyte differentiation into IL-10(high)IL-12absent dendritic cells with tolerogenic potential. J Immunol, 2005. 174(8): p. 4706-17.
70. Gansuvd, B., et al., Umbilical cord blood dendritic cells are a rich source of soluble HLA-DR: synergistic effect of exosomes and dendritic cells on autologous or allogeneic T-Cellproliferation. Hum Immunol, 2003. 64(4): p. 427-39.
71. Kim, S.H., et al., Exosomes derived ƒrom IL-10-treated dendritic cells can suppress inflammation and collagen-induced arthritis. J Immunol, 2005. 174(10): p. 6440-8.
72. Navarro, V., et al., The effect oƒ HLA class I (A and B) and class II (DR) compatibility on liver transplantation outcomes: an analysis of the OPTN database. Liver Transpl, 2006. 12(4): p. 652-8.
73. Gur, H., et al., Tolerance induction by megadose hematopoietic progenitor cells: expansion of veto cells by short-term culture of purified human CD34(+) cells. Blood, 2002. 99(11): p. 4174-81.
74. Reisner, Y., et al., Hematopoietic stem cell transplantation across major genetic barriers: tolerance induction by megadose CD34 cells and other veto cells. Ann N Y Acad Sci, 2003. 996: p. 72-9.
75. Donckier, V., et al., Donor stem cell infusion after non-myeloablative conditioning for tolerance induction to HLA mismatched adult living-donor liver graft. Transpl Immunol, 2004. 13(2): p. 139-46.
76. George, J.F., et al., An essential role for Fas ligand in transplantation tolerance induced by donor bone marrow. Nat Med, 1998. 4(3): p. 333-5.
77. Reisner, Y., et al., Crossing the HLA barriers. Blood Cells Mol Dis, 2004. 33(3): p. 206-10.
78. Kern, S., et al., Comparative analysis of mesenchymal stem cells from bone marrow, umbilical cord blood, or adipose tissue. Stem Cells, 2006. 24(5): p. 1294-301.
79. Deng, W., et al., Allogeneic bone marrow-derivedƒlk-1+Sca-1- mesenchymal stem cells leads to stable mixed chimerism and donor-specific tolerance. Exp Hematol, 2004. 32(9): p. 861-7.
80. Kadri, T., et al., Proteomic study of Galectin-1 expression in human mesenchymal stem cells. Stem Cells Dev, 2005. 14(2): p. 204-12.
81. Ryan, J.M., et al., Mesenchymal stem cells avoid allogeneic rejection. J Inflamm (Lond), 2005. 2: p. 8.
82. Beyth, S., et al., Human mesenchymal stem cells alter antigen-presenting cell maturation and induce T-cell unresponsiveness. Blood, 2005. 105(5): p. 2214-9.
83. Aggarwal, S. and M.F. Pittenger, Human mesenchymal stem cells modulate allogeneic immune cell responses. Blood, 2005. 105(4): p. 1815-22.
84. Plumas, J., et al., Mesenchymal stem cells induce apoptosis of activated T cells. Leukemia, 2005. 19(9): p. 1597-604.
85. Maccario, R., et al., Interaction of human mesenchymal stem cells with cells involved in alloantigen-specific immune response favors the differentiation of CD4+ T-cell subsets expressing a regulatory/suppressive phenotype. Haematologica, 2005. 90(4): p. 516-25.
86. Zappia, E., et al., Mesenchymal stem cells ameliorate experimental autoimmune encephalomyelitis inducing T-cell anergy. Blood, 2005. 106(5): p. 1755-61.
87. O’Donoghue, K., et al., Microchimerism in female bone marrow and bone decades after fetal mesenchymal stem-cell trafficking in pregnancy. Lancet, 2004. 364(9429): p. 179-82.
88. Halbrecht, J., Fresh and stored placental blood. Lancet, 1939. 2: p. 1263.
89. Hassall, O., et al., Umbilical-cord blood ƒor transfusion in children with severe anaemia in under-resourced countries. Lancet, 2003. 361(9358): p. 678-9.
90. Bhattacharya, N., Placental umbilical cord whole blood transfusion: a safe and genuine blood substitute for patients of the under-resourced world at emergency. J Am Coll Surg, 2005. 200(4): p. 557-63.
91. Bhattacharya, N., Placental umbilical cord whole blood transfusion to combat anemia in the background of tuberculosis and emaciation and its potential role as an immunoadjuvant therapy for the under-resourced people of the world. Clin Exp Obstet Gynecol, 2006. 33(2): p. 99-104.
92. Bhattacharya, N., Placental umbilical cord blood transfusion: A novel method of treatment of patients with malaria in the background of anemia. Clin Exp Obstet Gynecol, 2006. 33(1): p. 39-43.
93. Bhattacharya, N., Placental umbilical cord whole blood transfusion to combat anemia in the background of advanced rheumatoid arthritis and emaciation and its potential role as immunoadjuvant therapy. Clin Exp Obstet Gynecol, 2006. 33(1): p. 28-33.
94. Bhattacharya, N., A preliminary study of placental umbilical cord whole blood transfusion in under resourced patients with malaria in the background of anaemia. Malar J, 2006. 5: p. 20.
95. Bhattacharya, N., A preliminary report of 123 units of placental umbilical cord whole blood transfusion in HIV-positive patients with anemia and emaciation. Clin Exp Obstet Gynecol, 2006. 33(2): p. 117-21.
96. Bhattacharya, N., Spontaneous transient rise oƒ CD34 cells in peripheral blood after 72 hours in patients suffering from advanced malignancy with anemia: effect and prognostic implications of treatment with placental umbilical cord whole blood transfusion. Eur J Gynaecol Oncol, 2006. 27(3): p. 286-90.
97. Ito, K., et al., Possible mechanisms of immunotherapy for maintaining pregnancy in recurrent spontaneous aborters: analysis of anti-idiotypic antibodies directed against autologous T-cell receptors. Hum Reprod, 1999. 14(3): p. 650-5.
98. Smith, J.B., et al., The number of cells used ƒor immunotherapy of repeated spontaneous abortion influences pregnancy outcome. J Reprod Immunol, 1992. 22(3): p. 217-24.
99. Porter, D. and J.E. Levine, Graft-versus-host disease and graft-versus-leukemia after donor leukocyte infusion. Semin Hematol, 2006. 43(1): p. 53-61.
100. Szpakowski, A., et al., [The influence of paternal lymphocyte immunization on the balance of Th1/Th2 type reactivity in women with unexplained recurrent spontaneous abortion]. Ginekol Pol, 2000. 71(6): p. 586-92.
101. Hayakawa, S., et al., Effects of paternal lymphocyte immunization on peripheral Th1/Th2 balance and TCR V beta and V gamma repertoire usage of patients with recurrent spontaneous abortions. Am J Reprod Immunol, 2000. 43(2): p. 107-15.
102. Marleau, A.M. and N. Sarvetnick, T cell homeostasis in tolerance and immunity. J Leukoc Biol, 2005. 78(3): p. 575-84.
103. Hickman, S.P. and L.A. Turka, Homeostatic T cell proliferation as a barrier to T cell tolerance. Philos Trans R Soc Lond B Biol Sci, 2005. 360(1461): p. 1713-21.
104. Rosenberg, S.A. and M.E. Dudley, Cancer regression in patients with metastatic melanoma after the transfer of autologous antitumor lymphocytes. Proc Natl Acad Sci U S A, 2004. 101 Suppl 2: p. 14639-45.
105. Hess, A.D., et al., Specificity of effector T lymphocytes in autologous graft-versus-host disease: role of the major histocompatibility complex class II invariant chain peptide. Blood, 1997. 89(6): p. 2203-9.
106. Miura, Y., et al., Characterization of the T-cell repertoire in autologous graft-versus-host disease (GVHD): evidence for the involvement of antigen-driven T-cell response in the development of autologous GVHD. Blood, 2001. 98(3): p. 868-76.
107. Maeda, A., et al., Intravenous infusion of syngeneic apoptotic cells by photopheresis induces antigen-specific regulatory T cells. J Immunol, 2005. 174(10): p. 5968-76.
108. Lo, Y.M., et al., Two-way cell traffic between mother and ƒetus: biologic and clinical implications. Blood, 1996. 88(11): p. 4390-5.
109. Bianchi, D.W., et al., Male fetal progenitor cells persist in maternal blood ƒor as long as 27 years postpartum. Proc Natl Acad Sci USA, 1996. 93(2): p. 705-8.
110. Khosrotehrani, K. and D.W. Bianchi, Multi-lineage potential oƒ ƒetal cells in maternal tissue: a legacy in reverse. J Cell Sci, 2005. 118(Pt 8): p. 1559-63.
111. Johnson, K.L., et al., Significant fetal cell microchimerism in a nontransfused woman with hepatitis C: Evidence of long-term survival and expansion. Hepatology, 2002. 36(5): p. 1295-7.
112. Srivatsa, B., et al., Microchimerism of presumed fetal origin in thyroid specimens from women: a case-control study. Lancet, 2001. 358(9298): p. 2034-8.
113. Khosrotehrani, K., et al., Transfer oƒ ƒetal cells with multilineage potential to maternal tissue. Jama, 2004. 292(1): p. 75-80.
114. Khosrotehrani, K., et al., Combined FISH and immunolabeling on paraffin-embedded tissue sections for the study of microchimerism. Biotechniques, 2003. 34(2): p. 242-4.
115. Khosrotehrani, K. and D.W. Bianchi, Fetal cell microchimerism: helpful or harmful to the parous woman? Curr Opin Obstet Gynecol, 2003. 15(2): p. 195-9.
116. Wang, Y., et al., Fetal cells in mother rats contribute to the remodeling of liver and kidney after injury. Biochem Biophys Res Commun, 2004. 325(3): p. 961-7.
117. Tan, X.W., et al., Fetal microchimerism in the maternal mouse brain: a novel population oƒ ƒetalprogenitor or stem cells able to cross the blood-brain barrier? Stem Cells, 2005. 23(10): p. 1443-52.
118. Jinek, M., et al., A programmable dual-RNA-guided DNA endonuclease in adaptive bacterial immunity. Science, 2012. 337(6096): p. 816-21.
119. Cong, L., et al., Multiplex genome engineering using CRISPR/Cas systems. Science, 2013. 339(6121): p. 819-23.
120. Qi, L.S., et al., Repurposing CRISPR as an RNA-guidedplatform for sequence-specific control of gene expression. Cell, 2013. 152(5): p. 1173-83.
121. Nakamura, Y., T. Gojobori, and T. Ikemura, Codon usage tabulated from the international DNA sequence databases; its status 1999. Nucleic Acids Res, 1999. 27(1): p. 292.
122. Matheson, N.J., A.A. Peden, and P.J. Lehner, Antibody-ƒree magnetic cell sorting of genetically modified primary human CD4+ T cells by one-step streptavidin affinity purification. PLoS One, 2014. 9(10): p. e111437.
123. Meissner, T.B., et al., Genome editing for human gene therapy. Methods Enzymol, 2014. 546: p. 273-95.
124. Ebina, H., et al., A high excision potential of TALENsfor integrated DNA of HIV-based lentiviral vector. PLoS One, 2015. 10(3): p. e0120047.
125. Choi, Y.S. and S. Crotty, Retroviral vector expression in TCR transgenic CD4(+) T cells. Methods Mol Biol, 2015. 1291: p. 49-61.
126. Li, C., et al., Inhibition of HIV-1 infection of primary CD4+ T cells by gene editing of CCR5 using adenovirus-delivered CRISPR/Cas9. J Gen Virol, 2015.

What is claimed is:

1. A method of inducing an immune response to cancer-associated fibroblasts in a subject, the method comprising:
(a) isolating mononuclear cells from a cord blood sample;
(b) culturing the mononuclear cells with cancer-associated fibroblasts and one or more factors capable of enhancing cytotoxic activity of the mononuclear cells;
(c) inhibiting expression of an immune checkpoint protein in the mononuclear cells; and
(d) administering the mononuclear cells to the subject.

2. The method of claim 1, wherein the cancer-associated fibroblasts have been mitotically inactivated.

3. The method of claim 1 or 2, wherein (c) comprises RNA interference.

4. The method of claim 3, wherein (c) comprises providing to the mononuclear cells an antisense oligonucleotide capable of inhibiting expression of the immune checkpoint protein.

5. The method of claim 3 or 4, wherein (c) comprises providing to the mononuclear cells a short interfering RNA (siRNA) or short hairpin RNA (shRNA) capable of inhibiting expression of the immune checkpoint protein.

6. The method of any of claims 1-5, wherein the immune checkpoint protein is NR2F6, PD-1, PD-1L, TIM-3, CTLA-4, CD200, STAT6, or indolamine 2,3, deoxygenase (IDO).

7. The method of claim 6, wherein the immune checkpoint protein is PD-1, CTLA-4, or TIM-3.

8. The method of any of claims 1-7, wherein the one or more factors comprise one or more of IL-2, anti-CD3 antibodies, anti-CD28 antibodies, IL-7, IL-12, IL-17, IL-15, IL-18, and IL-33.

9. The method of claim 8, wherein the one or more factors comprise IL-2.

10. The method of claim 8, wherein the one or more factors comprise anti-CD3 antibodies and anti-CD28 antibodies.

11. The method of any of claims 1-10, wherein the one or more factors comprise a toll-like receptor (TLR) agonist.

12. The method of claim 11, wherein the TLR agonist is a TLR-1 agonist.

13. The method of claim 12, wherein the TLR-1 agonist is Pam3CSK4.

14. The method of claim 11, wherein the TLR agonist is a TLR-2 agonist.

15. The method of claim 14, wherein the TLR-2 agonist is heat-killed Listeria monocytogenes (HKLM).

16. The method of claim 11, wherein the TLR agonist is a TLR-3 agonist.

17. The method of claim 16, wherein the TLR-3 agonist is Poly I:C.

18. The method of claim 11, wherein the TLR agonist is a TLR-4 agonist.

19. The method of claim 18, wherein the TLR-4 agonist is Buprenorphine.

20. The method of claim 18, wherein the TLR-4 agonist is Carbamazepine.

21. The method of claim 18, wherein the TLR-4 agonist is Fentanyl.

22. The method of claim 18, wherein the TLR-4 agonist is Levorphanol.

23. The method of claim 18, wherein the TLR-4 agonist is Methadone.

24. The method of claim 18, wherein the TLR-4 agonist is Cocaine.

25. The method of claim 18, wherein the TLR-4 agonist is Morphine.

26. The method of claim 18, wherein the TLR-4 agonist is Oxcarbazepine.

27. The method of claim 18, wherein the TLR-4 agonist is Oxycodone.

28. The method of claim 18, wherein the TLR-4 agonist is Pethidine.

29. The method of claim 18, wherein the TLR-4 agonist is Glucuronoxylomannan from Cryptococcus.

30. The method of claim 18, wherein the TLR-4 agonist is Morphine-3-glucuronide.

31. The method of claim 18, wherein the TLR-4 agonist is lipoteichoic acid.

32. The method of claim 18, wherein the TLR-4 agonist is β-defensin 2.

33. The method of claim 18, wherein the TLR-4 agonist is small molecular weight hyaluronic acid.

34. The method of claim 18, wherein the TLR-4 agonist is fibronectin EDA.

35. The method of claim 18, wherein the TLR-4 agonist is snapin.

36. The method of claim 18, wherein the TLR-4 agonist is tenascin C.

37. The method of claim 11, wherein the TLR agonist is a TLR-5 agonist.

38. The method of claim 37, wherein the TLR-5 agonist is flagellin.

39. The method of claim 11, wherein the TLR agonist is a TLR-6 agonist.

40. The method of claim 39, wherein the TLR-6 agonist is FSL-1.

41. The method of claim 11, wherein the TLR agonist is a TLR-7 agonist.

42. The method of claim 41, wherein the TLR-7 agonist is imiquimod.

43. The method of claim 11, wherein the TLR agonist is a TLR-8 agonist.

44. The method of claim 43, wherein the TLR-8 agonist is ssRNA40/LyoVec.

45. The method of claim 11, wherein the TLR agonist is a TLR-9 agonist.

46. The method of claim 45, wherein the TLR-9 agonist is a CpG oligonucleotide.

47. The method of claim 45, wherein the TLR-9 agonist is ODN2006.

48. The method of claim 45, wherein the TLR-9 agonist is Agatolimod.

49. The method of any of claims 1-48, wherein (b) comprises culturing the mononuclear cells and the cancer-associated fibroblasts with an antigen presenting cell.

50. The method of claim 49, wherein the antigen presenting cell is a dendritic cell.

51. The method of claim 49, wherein the dendritic cell is a myeloid dendritic cell.

52. The method of claim 49, wherein the dendritic cell is a lymphoid dendritic cell.

53. The method of claim 49, wherein the antigen presenting cell is a B cell.

54. The method of claim 49, wherein the antigen presenting cell is a neutrophil.

55. The method of claim 49, wherein the antigen presenting cell is an endothelial cell.

56. The method of claim 49, wherein the antigen presenting cell is an artificial antigen presenting cell.

57. The method of any of claims 49-56, wherein the antigen presenting cell enhances immunogenicity of the mononuclear cells.

58. The method of claim 57, wherein the antigen presenting cell enhances immunogenicity of the mononuclear cells by augmenting HLA expression.

59. The method of claim 57, wherein the antigen presenting cell enhances immunogenicity of the mononuclear cells by augmenting TAP expression.

60. The method of claim 57, wherein the antigen presenting cell enhances immunogenicity of the mononuclear cells by augmenting CD80 expression.

61. The method of claim 57, wherein the antigen presenting cell enhances immunogenicity of the mononuclear cells by augmenting CD86 expression.

62. The method of claim 57, wherein the antigen presenting cell enhances immunogenicity of the mononuclear cells by augmenting IL-12 expression.

63. The method of any of claims 1-62, wherein (b) comprises culturing the mononuclear cells and the cancer-associated fibroblasts with a soluble inhibitor of one or more immunosuppressive factors.

64. The method of claim 63, wherein the soluble inhibitor is a small molecule.

65. The method of claim 63, wherein the soluble inhibitor is an antibody.

66. The method of any of claims 63-65, wherein the one or more immunosuppressive factors comprise one or more of HLA-G, IL-10, IDO, cyclo-oxygenases, and IL-20.

67. The method of any of claims 1-66, wherein, prior to (d), a chemotherapeutic agent is administered to the subject.

68. The method of claim 67, wherein the chemotherapeutic agent is a small molecule, a nucleic acid, an antibody, or an antibody-like molecule.

69. The method of claim 67 or 68, wherein the chemotherapeutic agent is ifosfamide, nimustine hydrochloride, cyclophosphamide, dacarbazine, melphalan, and ranimustine, gemcitabine hydrochloride, enocitabine, cytarabine ocfosfate, a cytarabine formulation, tegafur/uracil, a tegafur/gimeracil/oteracil potassium mixture, doxifluridine, hydroxycarbamide, fluorouracil, methotrexate, mercaptopurine, idarubicin hydrochloride, epirubicin hydrochloride, daunorubicin hydrochloride, daunorubicin citrate, doxorubicin hydrochloride, pirarubicin hydrochloride, bleomycin hydrochloride, peplomycin sulfate, mitoxantrone hydrochloride, mitomycin C, etoposide, irinotecan hydrochloride, vinorelbine tartrate, docetaxel hydrate, paclitaxel, vincristine sulfate, vindesine sulfate, vinblastine sulfate, anastrozole, tamoxifen citrate, toremifene citrate, bicalutamide, flutamide, estramustine phosphate, carboplatin, cisplatin, nedaplatin, thalidomide, neovastat, and bevacizumab, or L-asparaginase.

70. The method of any of claims 1-69, wherein the mononuclear cells are capable of inducing an immunological response in the subject toward the cancer-associated fibroblasts.

71. The method of claim 70, wherein the immunological response is cell proliferation.

72. The method of claim 70, wherein the immunological response is expression of CD69.

73. The method of claim 70, wherein the immunological response is expression of CD25.

74. The method of claim 70, wherein the immunological response is expression of perforin.

75. The method of claim 70, wherein the immunological response is expression of granzyme.

76. The method of claim 70, wherein the immunological response is expression of Fas ligand.

77. The method of claim 70, wherein the immunological response is expression of a cytokine that suppresses proliferation of tumor associated fibroblasts.

78. The method of claim 77, wherein the cytokine induces cell cycle arrest of tumor associated fibroblasts cells.

79. The method of claim 77, wherein the cytokine induces apoptosis of tumor associated fibroblast cells.

80. The method of claim 77, wherein the cytokine induces autophagy of tumor associated fibroblast cells.

81. The method of claim 77, wherein the cytokine induces necrosis of tumor associated fibroblast cells.

82. The method of claim 77, wherein the cytokine is interferon gamma.

83. The method of claim 77, wherein the cytokine is TNF-alpha, interferon alpha, interferon beta, interferon gamma, interferon omega, interferon tau, TRAIL, IL-2, IL-7, IL-12, IL-17, IL-18, IL-21, IL-22, IL-23, IL-27, IL-33, or HMGB1.

84. The method of any of claims 1-83, wherein the mononuclear cells are immune cells.

85. The method of claim 84, wherein the immune cells are natural killer (NK) cells.

86. The method of claim 84, wherein the immune cells are natural killer T (NKT) cells.

87. The method of claim 84, wherein the immune cells are T cells.

88. The method of claim 87, further comprising providing to the T cells a cancer-associated fibroblast antigen.

89. The method of claim 88, wherein the cancer-associated fibroblast antigen is fibroblast activated protein 1, FGF-9, TEM, VEGFR2, NA17, PDGFR-β, PAP, MAD-CT-2, Tie-2, PSA, protamine 2, legumain, endosialin, prostate stem cell antigen, carbonic anhydrase IX, STn, proteinase 3, GM3 ganglioside, or EpCAM.

90. The method of any of claims 87-89, wherein the T cells possess a Th1 phenotype.

91. The method of claim 90, wherein the T cells express one or more of CD4, CD94, CD119 (IFNγ R1), CD183 (CXCR3), CD186 (CXCR6), CD191 (CCR1), CD195 (CCR5), CD212 (IL-12Rβ1&2), CD254 (RANKL), CD278 (ICOS), IL-18R, MRP1, NOTCH3, and TIM3.

92. The method of any of claims 1-91, wherein, in (b), the mononuclear cells and the cancer-associated fibroblasts are further cultured with a histone deacetylase inhibitor.

93. The method of claim 92, wherein the histone deacetylase inhibitor is valproic acid.

94. A method of killing cancer-associated fibroblasts comprising culturing the cancer-associated fibroblasts with activated natural killer (NK) cells.

95. The method of claim 94, wherein the activated NK cells were obtained from cord blood.

96. The method of claim 94 or 95, wherein the activated NK cells comprise a genetic modification, wherein the genetic modification reduces the expression of an immune checkpoint protein.

97. The method of claim 96, wherein the immune checkpoint protein is NR2F6, PD-1, PD-1L, TIM-3, CTLA-4, CD200, STAT6, or indolamine 2,3, deoxygenase (IDO).

98. The method of any of claims 94-97, wherein the NK cells were activated using culture with IL-2.

99. The method of any of claims 94-98, further comprising culturing the cancer-associated fibroblasts and the activated NK cells with a histone deacetylase inhibitor.

100. The method of claim 99, wherein the histone deacetylase inhibitor is valproic acid.
